(19) United States
(12) Patent Application Publication
(10) Pub. No.: US 2021/0395709 A1
(43) Pub. Date: Dec. 23, 2021

(54) METHODS AND COMPOSITIONS INVOLVING THERMOSTABLE CAS9 PROTEIN VARIANTS

(71) Applicants: Chan Zuckerberg Biohub, Inc., San Francisco, CA (US); The Board of Trustees of the Leland Stanford Junior University, Stanford, CA (US)

(72) Inventors: Stephen R. Quake, San Francisco, CA (US); Stephanie Tzouanas Schmidt, Stanford, CA (US); Feiqiao Brian Yu, San Francisco, CA (US); Paul Blainey, Stanford, CA (US); Andrew Paul May, San Francisco, CA (US)

(21) Appl. No.: 17/285,660

(22) PCT Filed: Oct. 17, 2019
(86) PCT No.: PCT/US2019/056730

§ 371 Date: Apr. 15, 2021

Related U.S. Application Data
(60) Provisional application No. 62/901,495, filed on Sep. 17, 2019.
(60) Provisional application No. 62/747,619, filed on Oct. 18, 2018.

(51) Int. Cl. C12N 9/22; C12N 15/11; C12N 15/90

(52) U.S. Cl. CPC C12N 9/22; C12N 15/11; C12N 2800/80; C12N 2310/20; C12N 15/907

(57) ABSTRACT

The disclosure provides Cas9 protein variants that are thermostable at elevated temperatures (e.g., at least 70° C. or above). A Cas9 protein may have at least 75% sequence identity to a wild-type Cas9 protein (e.g., a wild-type Cas9 protein having the sequence of SEQ ID NO:1) and/or one or more amino acid substitutions relative to the wild-type Cas9 protein.

### CROSS-REFERENCE TO RELATED APPLICATIONS

This application claims priority to U.S. Provisional Application No. 62/747,619, filed Oct. 18, 2018, and U.S. Provisional Application No. 62/901,495, filed Sep. 17, 2019, the disclosures of which are hereby incorporated by reference in their entireties for all purposes.

### BACKGROUND OF THE INVENTION

The application of clustered regularly interspaced short palindromic repeats (CRISPR) and CRISPR-associated (Cas) proteins has revolutionized molecular biology by making genome editing possible in both prokaryotes and eukaryotes (Jinek, Cong). Constituting the heritable and adaptive immune system of prokaryotes, CRISPR-Cas9 systems are present in archaea and bacteria from diverse environments (Koonin). A wide variety of CRISPR-Cas9 systems exist, and Class 2 systems, particularly type II systems, have been well characterized and broadly implemented in part because these systems rely on a single effector protein, Cas9, and an RNA duplex, which can be replaced by a single-guide RNA (sgRNA). CRISPR-Cas9 systems, particularly that from Streptococcus pyogenes, have been leveraged to edit genomes across organisms and create new tools for sequencing applications (Wang). Nearly all Cas9 proteins have been derived from mesophilic hosts, making their use in applications requiring elevated temperatures and robust stability difficult. Improved materials and methods for carrying out gene editing especially in challenging environments with high temperatures are needed.

The CRISPR-Cas nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” This system has now been engineered such that the crRNA and tracrRNA can be combined into one molecule (the “single-guide RNA” or “sgRNA”), and the crRNA equivalent portion of the single-guide RNA can be engineered to guide the Cas (e.g., Cas9) nuclease to target any desired sequence.

Target identification relies first on identification of the protospacer adjacent motif (PAM) sequence located downstream of the target sequence, and then RNA-DNA Watson-Crick hybridization between an approximately 20-nucleotide stretch of the sgRNA and the DNA target site. After an allosteric change induced by sgRNA hybridization to the target DNA, Cas9 is triggered to cleave both target DNA strands creating a blunt-end double-strand break. Double-strand break formation activates one of two highly conserved repair mechanisms, canonical non-homologous end-joining (NHEJ) and homology-directed repair (HDR) (e.g., homologous recombination (HR)). Thus, the CRISPR-Cas system can be engineered to create a double-strand break at a desired target in a genome of a cell, and harness the cell's endogenous mechanisms to repair the induced break by HDR or NHEJ.

Previously, two Cas9 proteins from thermophiles have been reported, providing enhanced stability in in vivo environments and enabling genome editing in thermophilic organisms (Harrington et al., Nature Communications 8(1):1424, 2017 and Mougiakos et al., Nature Communications 8(1):1647, 2017). These two proteins, GeoCas9 and ThermoCas9, were identified by sequencing environmental samples, and their hosts live at temperatures of 65° C. and 70° C., respectively. GeoCas9 is a thermostable Cas9 protein from Geobacillus stearothermophilus. GeoCas9 maintains activity over a temperature range of between 45° C. and 70° C. By harnessing the natural sequence variation of GeoCas9 from closely related species, a PAM variant was engineered that recognizes additional PAM sequences and thereby doubles the number of targets accessible to this system. A highly efficient single-guide RNA (sgRNA) was also made for GeoCas9 using RNA-seq data from the native organism. GeoCas9, together with is sgRNA, was demonstrated to efficiently edit genomic DNA in mammalian cells (Harrington et al., Nature Communications 8(1):1424, 2017). ThermoCas9 is a DNA endonuclease from the CRISPR-Cas type II-C system of the thermophilic bacterium Geobacillus thermodenitrificans T1230. ThermoCas9 is active in vitro between 37° C. and 70° C. The PAM preferences of ThermoCas9 are very strict for activity in the lower part of the temperature range, whereas more variety in the PAM is allowed for activity at the moderate to optimal temperatures (37-60° C.) (Mougiakos et al., Nature Communications 8(1):1647, 2017). ThermoCas9-based engineering tools for gene deletion and transcriptional silencing at 55° C. in Bacillus smithii and for gene deletion at 37° C. in Pseudomonas putida were developed (Mougiakos et al., Nature Communications 8(1):1647, 2017).

### SUMMARY OF THE INVENTION

In one aspect, the disclosure features an isolated clustered regularly interspaced short palindromic repeats (CRISPR)-associated (Cas) 9 protein variant comprising the sequence of SEQ ID NO: 1 or an enzymatically active variant or fragment thereof, wherein the enzymatically active variant or fragment has Cas9 nuclease activity at 70° C. or above.

In one aspect, the disclosure features an isolated Cas9 protein variant comprising a sequence having at least 75% sequence identity to the sequence of a wild-type Cas9 protein, wherein the Cas protein variant has at least one amino acid substitution relative to the sequence of the wild-type Cas9 protein, and wherein the wild-type Cas9 protein has a sequence of SEQ ID NO:1.

In some embodiments, the isolated Cas9 protein variant is a fragment of the wild-type Cas9 protein. In some embodiments, the isolated Cas9 protein variant has nuclease activity at a temperature of between 20° C. and 100° C. In some embodiments, the isolated Cas9 protein variant has nuclease activity at a temperature of at least 70° C.

In some embodiments, the isolated Cas9 protein variant forms a ribonucleoprotein complex with a single-guide RNA (sgRNA), wherein the sgRNA comprises a guide sequence and a scaffold sequence.

In some embodiments, the guide sequence has at least 22 nucleotides (e.g., between 22 and 25 nucleotides). In some embodiments, the scaffold sequence has at least 75% sequence identity to the sequence of GUUGUGAUUUGCUUUCAAAGAAAUUUGAAGCAAAUCACAAUAAGGAUUUUUCCGUUGUGAAAACAUU UACAGUAGUCCCGAUGCAAACCAUCGGGAUUGUUGUUUU (SEQ ID NO:7).

In some embodiments, the isolated Cas9 protein variant recognizes an adenine-rich protospacer adjacent motif (PAM) sequence. The adenine-rich PAM sequence may comprise at least 40% adenine in its sequence. In particular embodiments, the adenine-rich PAM sequence has at least 70% sequence identity to the sequence of CCACATCGAA (SEQ ID NO:4) or AGACATGAAA (SEQ ID NO:5).

In some embodiments, the isolated Cas9 protein variant binds a PAM motif having the sequence of NVRNAT (SEQ ID NO:6), wherein N is any nucleotide, V is A, G or C, and R is G or A. In some embodiments, the isolated Cas9 protein variant binds a PAM motif having the sequence of NRRNAT (SEQ ID NO:13), wherein N is any nucleotide and R is G or A. In particular embodiments, the PAM motif has the sequence of GGACAT (SEQ ID NO:10).

In another aspect, the disclosure features a ribonucleoprotein complex comprising:

(1) an isolated Cas9 protein variant comprising a sequence having at least 75% sequence identity to the sequence of a wild-type Cas9 protein, and
(2) an sgRNA comprising a guide sequence and a scaffold sequence,
wherein the scaffold sequence has at least 75% sequence identity to the sequence of SEQ ID NO:7.

In another aspect, the disclosure features a composition comprising:

(1) a ribonucleoprotein complex comprising:
(a) an isolated Cas9 protein variant comprising a sequence having at least 75% sequence identity to the sequence of a wild-type Cas9 protein, and
(b) an sgRNA comprising a guide sequence and a scaffold sequence, and
   (2) a ribosomal complementary DNA (cDNA),
   wherein the scaffold sequence has at least 75% sequence identity to the sequence of SEQ ID NO:7.

In some embodiments of this aspect, the ribosomal cDNA is generated in a polymerase chain reaction (PCR).

In some embodiments of the previous two aspects, the isolated Cas9 protein variant comprises at least one amino acid substitution relative to the sequence of the wild-type Cas9 protein. In some embodiments, the isolated Cas9 protein variant comprises a fragment of the wild-type Cas9 protein. In some embodiments, the wild-type Cas9 protein has the sequence of SEQ ID NO:1.

In some embodiments of the previous two aspects, the isolated Cas9 protein variant has nuclease activity at a temperature of between 20° C. and 100° C. In some embodiments, the isolated Cas9 protein variant has nuclease activity at a temperature of at least 70° C.

In some embodiments of the previous two aspects, the isolated Cas9 protein variant recognizes an adenine-rich protospacer adjacent motif (PAM) sequence. The adenine-rich PAM sequence comprises at least 40% adenine in its sequence. In some embodiments, the adenine-rich PAM sequence has at least 70% sequence identity to the sequence of CCACATCGAA (SEQ ID NO:4) or AGACATGAAA (SEQ ID NO:5). In other embodiments, the isolated Cas9 protein variant recognizes a PAM sequence having the sequence of NVRNAT (SEQ ID NO:6), wherein N is any nucleotide, V is A, G or C, and R is G or A. In other embodiments, the isolated Cas9 protein variant recognizes a PAM sequence having the sequence of NRRNAT (SEQ ID NO:13), wherein N is any nucleotide and R is G or A.

In another aspect, the disclosure features a cell comprising a ribonucleoprotein complex described herein.

In another aspect, the disclosure features a method of altering the genome of a cell, comprising contacting the cell with:

(1) an isolated Cas9 protein variant comprising a sequence having at least 75% sequence identity to the sequence of a wild-type Cas9 protein, and
(2) an sgRNA comprising a guide sequence and a scaffold sequence,
wherein the scaffold sequence has at least 75% sequence identity to the sequence of SEQ ID NO:7,
wherein the isolated Cas9 protein variant interacts with the sgRNA and a target DNA within the cell, and
wherein the guide sequence in the sgRNA comprises a region complementary to a region of the target DNA.

In some embodiments of this aspect, the isolated Cas9 protein variant recognizes an adenine-rich PAM sequence. The adenine-rich PAM sequence may comprise at least 40% adenine in its sequence. In particular, the adenine-rich PAM sequence may have at least 70% sequence identity to the sequence of CCACATCGAA (SEQ ID NO:4) or AGACATGAAA (SEQ ID NO:5). In some embodiments, the isolated Cas9 protein variant recognizes a PAM sequence having the sequence of NVRNAT (SEQ ID NO:6), wherein N is any nucleotide, V is A, G or C, and R is G or A. In some embodiments, the isolated Cas9 protein variant recognizes a PAM sequence having the sequence of NRRNAT (SEQ ID NO:13), wherein N is any nucleotide and R is G or A.

### BRIEF DESCRIPTION OF THE DRAWINGS

FIG. 1A shows a phylogenetic tree of representative Cas9 proteins from type II systems.

FIG. 1B shows architectural domains of IgnaviCas9 and SpyCas9 where REC is the recognition lobe.

FIG. 1C shows a homology model of IgnaviCas9 with the domains annotated. The model was generated using Phyre2.

FIG. 2A shows a representation of the determined sgRNA with important structural features labeled.

FIG. 2B shows testing of the preferred spacer length was conducted by comparing cleavage at 52° C. of templates targeted by truncated versions of the initial spacer. The cut-to-uncut-ratio was normalized to that corresponding to 25 nt (length used for preliminary experiments).

FIG. 3A shows an electropherogram showing cleavage of template containing the PAM from P. lavamentivorans compared to a control reaction with scrambled sgRNA and to the sgRNA from the experimental condition.

FIG. 3B shows nucleic acid logo results from sequences flanking the IgnaviCas9 CRISPR array spacers identified from bulk sequencing of the environmental sample from which IgnaviCas9 was identified.

FIG. 3C shows the performance of IgnaviCas9 in cleaving DNA templates with the indicated substitutions at the specified positions for the starting sequence of AGACAT (SEQ ID NO:12). Substitutions abolishing cleavage activity enabled PAM refinement.

FIG. 3D shows an electropherogram showing cleavage of template containing the PAM from P. lavamentivorans with adjustments informed by leads from bulk sequencing data. Curves from control reaction with scrambled sgRNA and from experimental condition sgRNA are included for comparison.

FIG. 4A shows a bar graph showing the efficiency of IgnaviCas9 in cleaving DNA templates compared over a range of temperatures. The average and standard deviation at each temperature tested is shown (n=3).

FIG. 4B shows a bar graph showing the upper temperature limit of Cas9 homologs.

FIG. 4C shows a scatterplot showing IgnaviCas9's rate of DNA cleavage compared to that of SpyCas9 over a range of temperatures.

FIG. 5 shows the alignment of the amino acid sequences of several Cas proteins.

FIG. 6 shows the reduction of targeted sequence by IgnaviCas9. Coverage plot for 16s rRNA sequence targeted by IgnaviCas9 during PCR amplification. Normalized coverage given as per-base coverage divided by average whole genome coverage.

### DETAILED DESCRIPTION OF THE INVENTION

### 1. Definitions

As used herein, the term “Cas9 protein variant” refers to a protein that has Cas9 nuclease activity at elevated temperatures, e.g., above 70° C. (e.g., 72° C., 75° C., 77° C., 80° C., 82° C., 85° C., 87° C., 90° C., 92° C., 95° C., 97° C., or 100° C.). In some embodiments, a Cas9 protein variant has at least 75% sequence identity (e.g., at least 75%, 80%, 85%, 90%, 91%, 92%, 93%, 94% 95%, 96%, 97%, 98%, 99%, or 100% sequence identity) to the sequence of a wild-type (WT) Cas9 protein having the sequence of SEQ ID NO:1. In some embodiments, the Cas9 protein variant is an isolated protein that has the sequence of SEQ ID NO:1. In some embodiments, the Cas9 protein variant has at least one amino acid substitution (e.g., one, two, three, four, five, six, seven, eight, nine, ten, or more amino acid substitutions) relative to the sequence of SEQ ID NO:1. A Cas9 protein variant may also be a protein that is a truncated version or fragment of a wild-type Cas9 protein having the sequence of SEQ ID NO:1. Further, a Cas9 protein variant may be a fragment of the wild-type Cas9 protein having the sequence of SEQ ID NO:1 and have at least one amino acid substitution relative to the sequence of SEQ ID NO:1.

As used herein, the term “fragment” or “truncated version” refers to a portion of a protein. A truncated version or fragment of a wild-type Cas9 protein (e.g., a wild-type Cas9 protein having the sequence of SEQ ID NO:1) refers to a Cas9 protein variant that has at least 50 contiguous amino acids (e.g., 50, 100, 150, 200, 250, 300, 350, 400, 450, 500, 550, 600, 650, 700, 750, 800, 850, 900, 950, 1000, 1050, 1100, 1150, 1200, 1210, 1220, 1230, or 1235 contiguous amino acids) of the wild-type Cas9 protein.

As used herein, the term “single-guide RNA” or “sgRNA” refers to a DNA-targeting RNA containing a guide sequence (i.e., crRNA equivalent portion of the single-guide RNA) that targets the Cas protein to the target DNA and a scaffold sequence (i.e., tracrRNA equivalent portion of the single-guide RNA) that interacts with the Cas protein.

As used herein, the term “ribonucleoprotein complex” refers to a complex comprising a Cas9 Protein or variant and RNA. The ribonucleic acid complex may comprise an sgRNA and a Cas9 protein or variant, or, alternatively, a Cas9 protein or variant, a crRNA and a tracrRNA).

As used herein, the term “adenine-rich protospacer adjacent motif (PAM) sequence” refers to a PAM sequence that has at least 40% adenine. As described further herein, in some embodiments, a Cas9 protein variant recognizes an adenine-rich PAM sequence located downstream of the target DNA. An adenine-rich PAM sequence may be CCACATCGAA (SEQ ID NO:4) or AGACATGAAA (SEQ ID NO:5).

As used herein, the term “percent (%) sequence identity” refers to the percentage of amino acid residues or nucleic acid bases of a candidate sequence, e.g., a Cas9 protein variant, that are identical to the amino acid (or nucleic acid) residues of a reference sequence, e.g., a wild-type Cas9 protein, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art and is described in detail in section 2.4.

### 2. Introduction

Disclosed herein are compositions and methods directed to a CRISPR-Cas9 system from a hyperthermophilic Ignavibacterium discovered using mini-metagenomic sequencing from the Yellowstone National Parks Lower Geyser Basin in which temperatures average 90° C.

### 2.1 Wild-Type IgnaviCas9

IgnaviCas9 is a type II-C Cas9 protein from a hyperthermophilic Ignavibacterium identified through mini-metagenomic sequencing of samples from a hot spring. IgnaviCas9 has nuclease activity at temperatures up to 100° C. in vitro, which enables genome editing beyond the 44° C. limit of Streptococcus pyogenes Cas9 (SpyCas9) and the 70° C. limit of both Geobacillus stearothermophilus Cas9 (GeoCas9) and Geobacillus thermodenitrificans T12 Cas9 (ThermoCas9). A wild-type IgnaviCas9 protein has the amino acid sequence of SEQ ID NO:1, which is encoded by the nucleic acid sequence of SEQ ID NO:2. SEQ ID NO:3 is a codon-optimized nucleic acid sequence encoding the wild-type protein for expression in E coli.

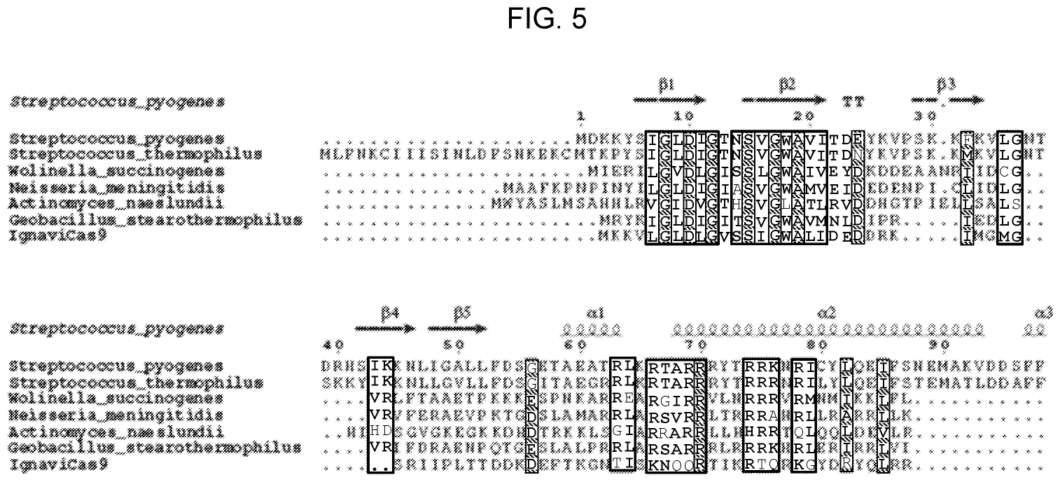

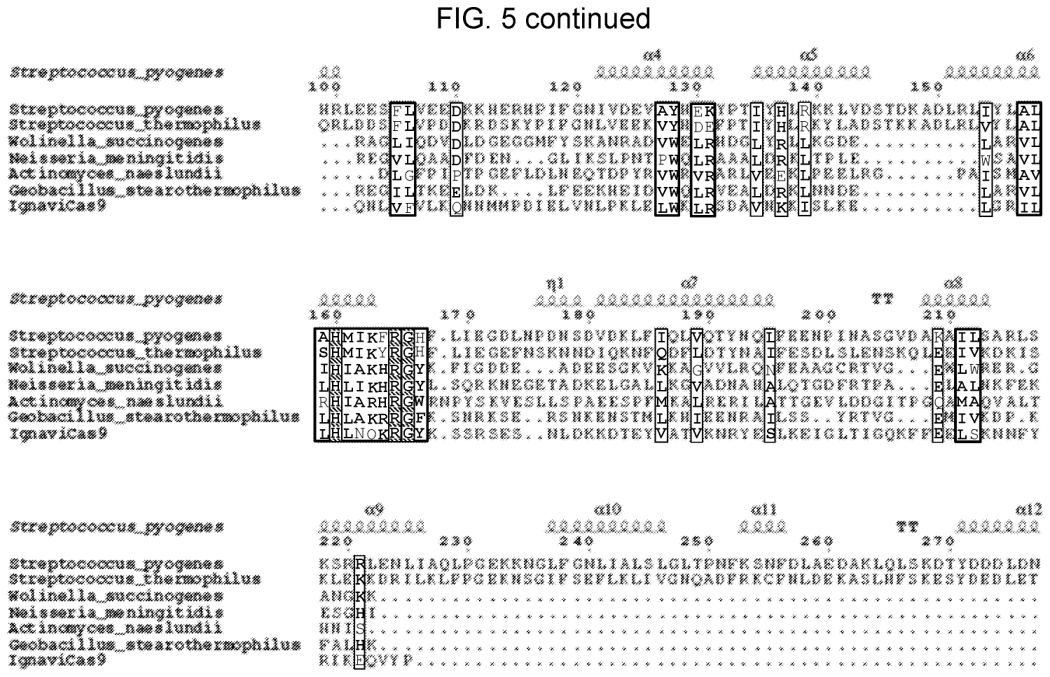

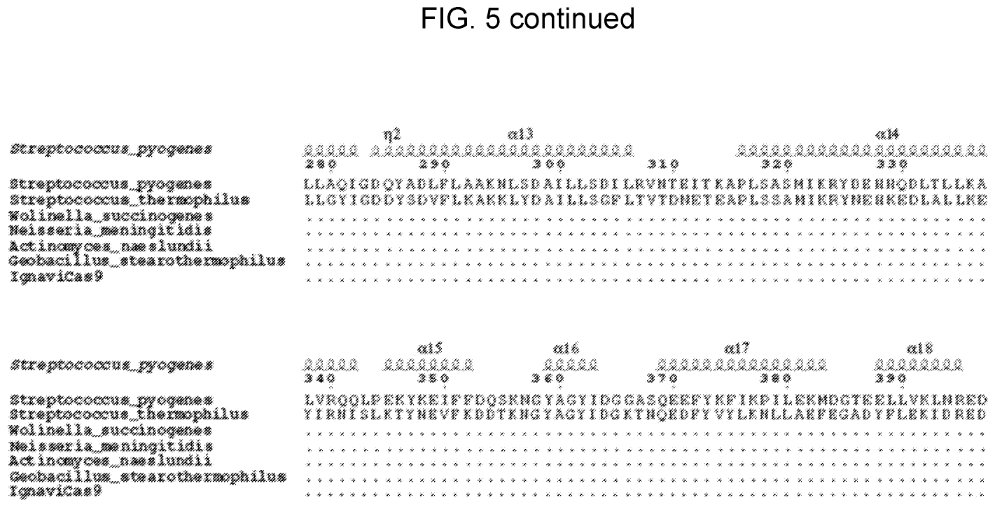

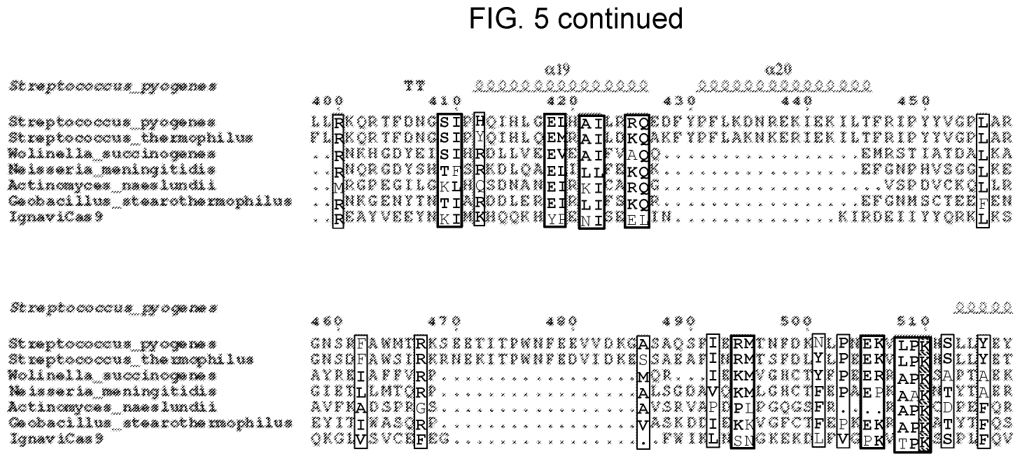

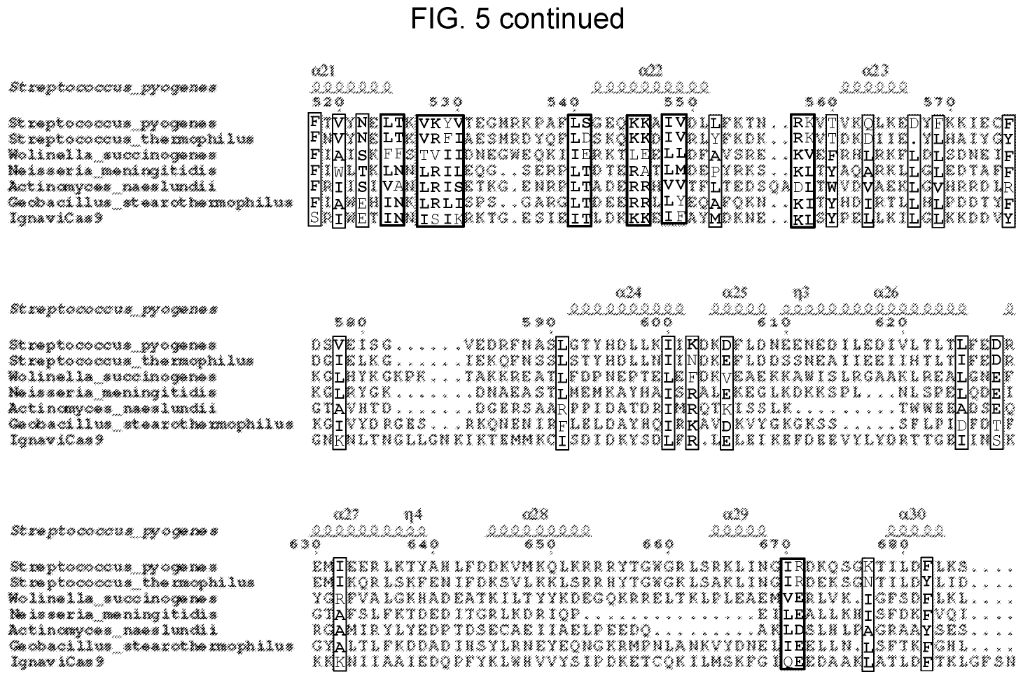

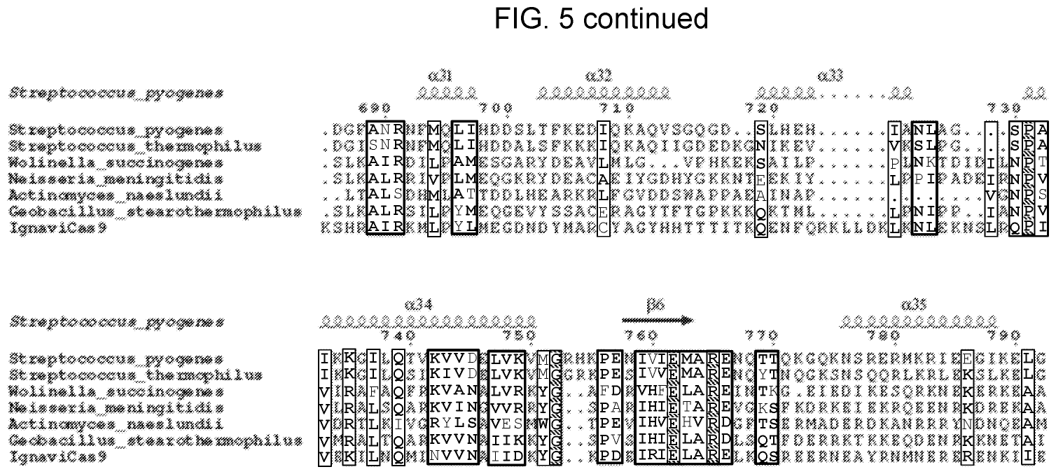

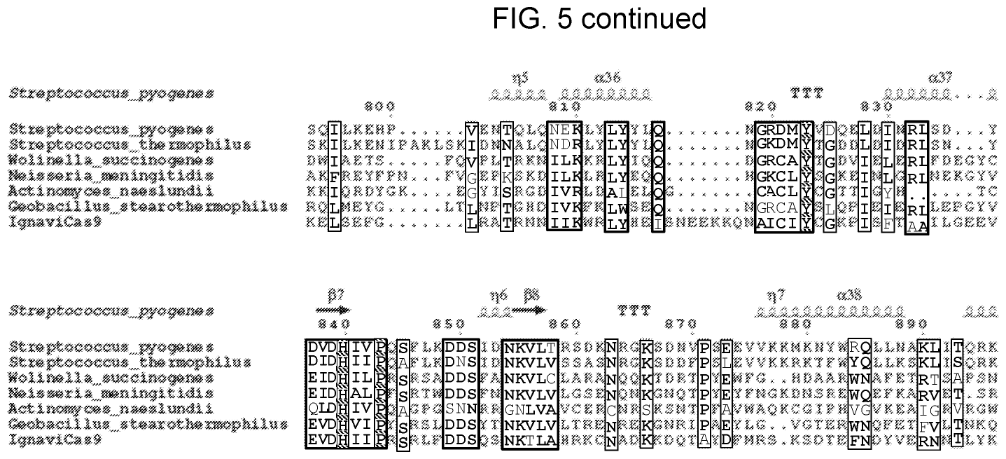

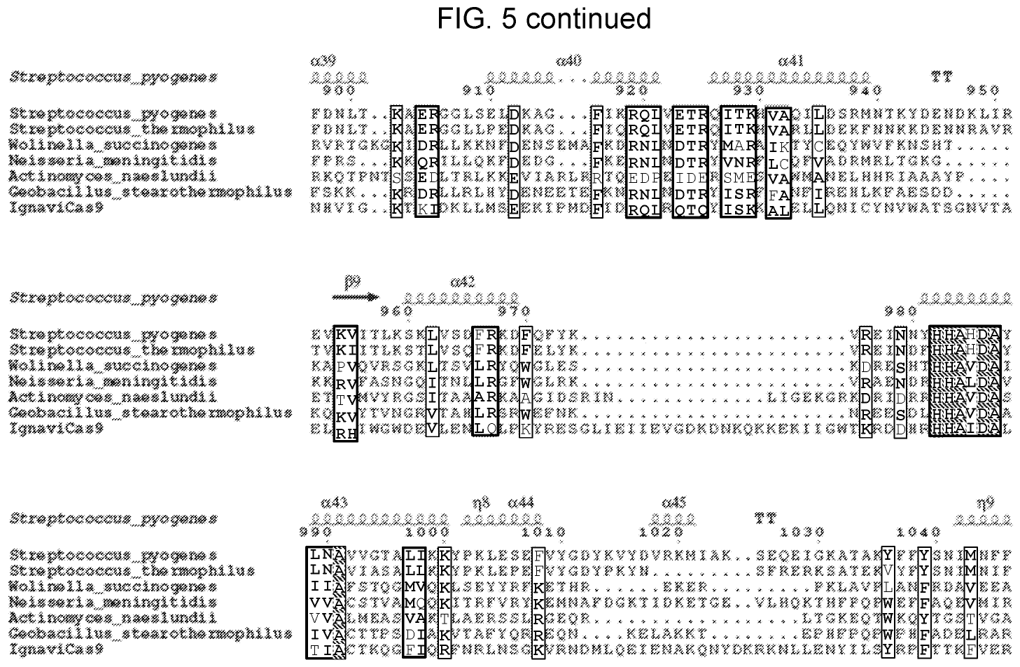

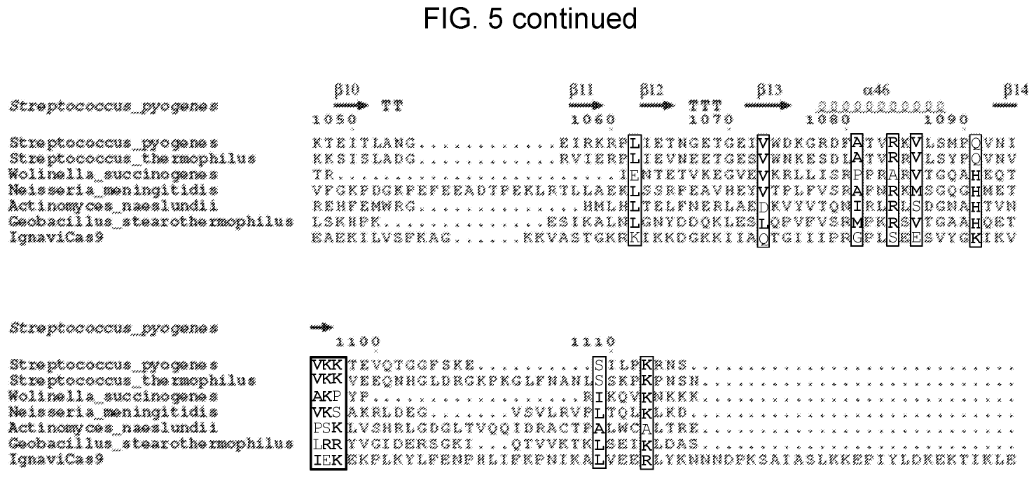

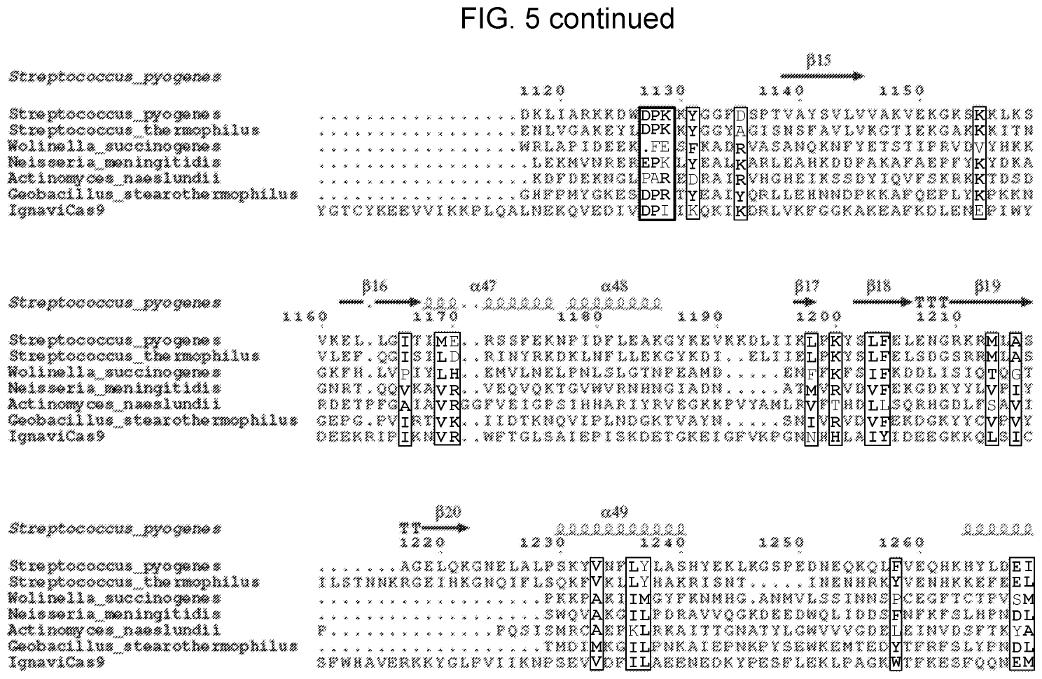

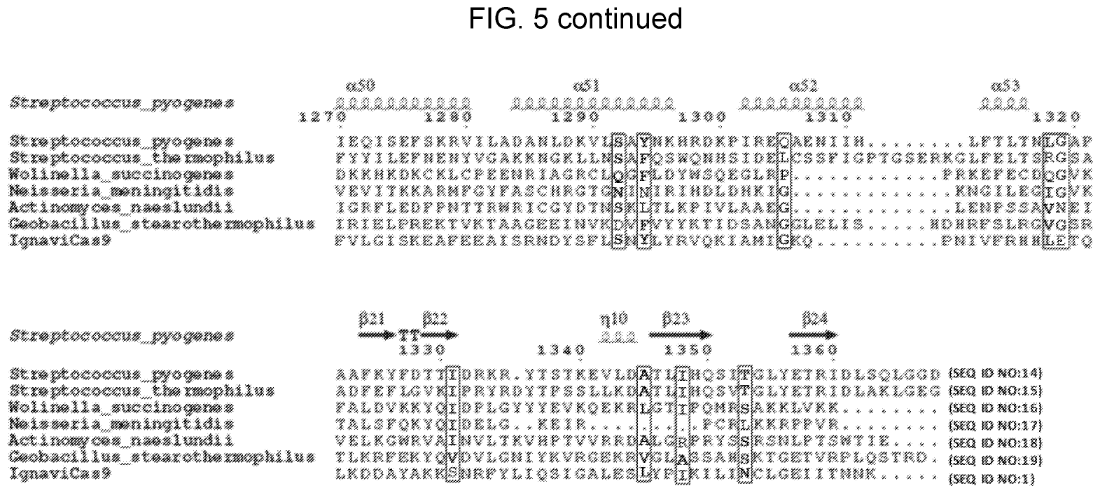

FIG. 5 shows a sequence alignment of IgnaviCas9 with several other Cas proteins. The following amino acid positions are conserved: Gly at position 6, Asp at position 8, Gly at position 10, Ser at positon 13, Gly at position 15, Ala at position 17, Arg at position 56, His at position 122, Arg at position 127, Gly at position 128, Lys at position 264, Pro at position 506, Gly at position 527, Glu at position 535, Arg at position 538, Tyr at position 602, His at position 622, Pro at position 625, His at position 789, His at position 790, Ala at position 791, Asp at position 793, Ala at position 794, and Ala at position 798.

### 2.2 IgnaviCas9 Variants

The disclosure features a Cas9 protein variants with at least at least 75% sequence identity (e.g., at least 75%, 80%, 85%, 90%, 91%, 92%, 93%, 94% 95%, 96%, 97%, 98%, 99%, or 100% sequence identity) to the sequence of a wild-type Cas9 protein (e.g., a wild-type Cas9 protein having a sequence of SEQ ID NO:1). In one approach the Cas9 variant is enzymatically active. Enzymatic activity may be measured as described below in §§ 2.5 and 2.6 and Example 4, or using art known assays.

In one approach, a Cas9 protein variant has Cas9 nuclease activity at 20° C. In one approach, a Cas9 protein variant has Cas9 nuclease activity at 40° C. In one approach, a Cas9 protein variant has Cas9 nuclease activity at 60° C. In one approach, a Cas9 protein variant has Cas9 nuclease activity at 80° C. In one approach, a Cas9 protein variant has Cas9 nuclease activity at 90° C. A Cas9 protein variant has Cas9 nuclease activity at elevated temperatures, e.g., from 20 to 90° C., e.g., or from 20° C. to 100° C. (e.g., from 25° C. to 100° C., from 30° C. to 100° C., from 35° C. to 100° C., from 40° C. to 100° C., from 45° C. to 100° C., from 50° C. to 100° C., from 55° C. to 100° C., from 60° C. to 100° C., from 65° C. to 100° C., from 70° C. to 100° C., from 75° C. to 100° C., from 80° C. to 100° C., from 85° C. to 100° C., from 90° C. to 100° C., or from 95° C. to 100° C.; e.g., 20° C., 25° C., 30° C., 35° C., 40° C., 45° C., 50° C., 55° C., 60° C., 65° C., 70° C., 75° C., 80° C., 85° C., 90° C., 95° C., or 100° C.). In some embodiments, the Cas9 protein variant has nuclease activity at temperatures above 70° C. (e.g., 72° C., 75° C., 77° C., 80° C., 82° C., 85° C., 87° C., 90° C., 92° C., 95° C., 97° C., or 100° C.).

In some embodiments, a Cas9 protein variant may have one, two, three, four, five, six, seven, eight, nine, ten, or more amino acid substitutions relative to a wild-type Cas9 protein (e.g., a wild-type Cas9 protein having the sequence of SEQ ID NO:1). In some embodiments, a Cas9 protein variant as disclosed herein may be a truncated version or fragment of a wild-type Cas9 protein, e.g., a truncated version or fragment of a wild-type Cas9 protein having the sequence of SEQ ID NO:1. A Cas9 protein variant that is a truncated version or fragment of the wild-type Cas9 protein having the sequence of SEQ ID NO:1 may comprise at least 50 contiguous amino acids (e.g., 50, 100, 150, 200, 250, 300, 350, 400, 450, 500, 550, 600, 650, 700, 750, 800, 850, 900, 950, 1000, 1050, 1100, 1150, 1200, 1210, 1220, 1230, or 1235 contiguous amino acids). In further embodiments, a Cas9 protein variant may be a fragment of a wild-type Cas9 protein (e.g., a wild-type Cas9 protein having the sequence of SEQ ID NO:2) and have one, two, three, four, five, six, seven, eight, nine, ten, or more amino acid substitutions relative to the wild-type Cas9 protein.

A Cas9 protein variant as disclosed herein may include one of more (e.g. all) of the following conserved amino acids (see, e.g., FIG. 5): Gly at position 6, Asp at position 8, Gly at position 10, Ser at positon 13, Gly at position 15, Ala at position 17, Arg at position 56, His at position 122, Arg at position 127, Gly at position 128, Lys at position 264, Pro at position 506, Gly at position 527, Glu at position 535, Arg at position 538, Tyr at position 602, His at position 622, Pro at position 625, His at position 789, His at position 790, Ala at position 791, Asp at position 793, Ala at position 794, and Ala at position 798, wherein the amino acid positions are numbered with reference to SEQ ID NO:1. In other words, the amino acid substitution(s) in the Cas9 protein variant relative to a wild-type Cas9 protein (e.g., a wild-type Cas9 protein having the sequence of SEQ ID NO:1) are not at any of the amino acid positions listed above.

### 2.3 PAM Specificity

As described in Example 1, a PAM optimally recognized by the WT IgnaviCas9 is NVRNAT (SEQ ID NO:6). In some embodiments, the isolated Cas9 protein variant recognizes a PAM sequence having the sequence of NRRNAT (SEQ ID NO:13). In some embodiments, the Cas9 protein variant disclosed herein recognizes adenine-rich PAM sequences, such as CCACATCGAA (SEQ ID NO:4) and AGACATGAAA (SEQ ID NO:5). In some embodiments, the Cas9 protein variant disclosed herein recognizes an adenine-rich PAM sequence having at least 70% sequence identity (e.g., 70%, 80%, 90%, or 100% sequence identity) to the sequence of SEQ ID NO:4 or 5. In other embodiments, the Cas9 protein variant disclosed herein recognizes the PAM motif NVRNAT (SEQ ID NO:6), where N is any nucleotide (e.g., A, T, C, or G), V is A, G or C, and R is G or A. In other embodiments, the Cas9 protein variant disclosed herein recognizes the PAM motif NRRNAT (SEQ ID NO:13), where N is any nucleotide (e.g., A, T, C, or G) and R is G or A. In yet other embodiments, the Cas9 protein variant disclosed herein recognizes the PAM sequence GGACAT (SEQ ID NO:10).

A target DNA sequence (e.g., a target DNA sequence having 22 to 25 nucleotides) recognized and cleaved by a Cas9 protein variant described herein may be followed by a PAM sequence having at least 70% sequence identity (e.g., 70%, 80%, 90%, or 100% sequence identity) to the sequence of SEQ ID NO:4 or 5. A target DNA sequence (e.g., a target DNA sequence having 22 to 25 nucleotides) recognized and cleaved by a Cas9 protein variant described herein may also be followed by the PAM sequence of SEQ ID NO:6.

### 2.4 Determination of Sequence Identity

A number of methods and tools are available to determine and compare the percent sequence identity between a Cas9 protein variant and a wild-type Cas9 protein (e.g., the sequence of SEQ ID NO:1). For sequence comparison, typically one sequence acts as a reference sequence (e.g., the sequence of a wild-type Cas9; SEQ ID NO:1), to which test sequences are compared (e.g., the sequence of a Cas9 protein variant).

In one approach a variant is aligned with SEQ ID NO:1 to maximize amino acid residue identities. In this approach the % identity can be the number of identities (where a gap is considered a nonidentity) divided by 1240.

Common computer-implemented sequence comparison algorithms are used to determine sequence identity. When using a sequence comparison algorithm (e.g., BLAST), test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. in which a sequence may be compared to a reference sequence of the same number of contiguous positions after the two sequences are optimally aligned.

Algorithms that are suitable for determining percent sequence identity and sequence similarity are available in the art, e.g., BLAST. Software for performing BLAST analyses (see, e.g., Altschul et al. (1990) J. Mol. Biol. 215: 403-410 and Altschul et al. (1977) Nucleic Acids Res. 25: 3389-3402) is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10-5, and most preferably less than about 10-20.

### 2.5 Cas9 Nuclease Activity

In some embodiments, the Ignavibacterium Cas9 variants and fragments described herein have Cas9 nuclease activity. Typically the Ignavibacterium Cas9 variants and fragments described herein have Cas9 nuclease activity at elevated temperature (e.g., above 70° C., above 80° C., or above 90° C.). In vitro assays for Cas9 activity are well known (see, e.g., Anders and Jinek, Methods in Enzymology 546:1-20, 2014). In one approach, a ribonucleoprotein complex comprising the Cas9 protein or variant and an sgRNA (e.g., an sgRNA having the sequence of SEQ ID NO:9) is combined with a target DNA substrate e.g., SEQ ID NO.8, which comprises the DNA target sequence GGGAATAGTTACATTACTATCTGTA (SEQ ID NO:11) under assay conditions described below in Example 4 except that the assay temperature may be selected for 37°, 70°, 80°, or 90° C.

### 2.6 Assays for Thermal Stability

A Cas9 protein variant as disclosed herein is thermostable in a wide temperature range, i.e., from 20° C. to 100° C. (e.g., 20° C., 25° C., 30° C., 35° C., 40° C., 45° C., 50° C., 55° C., 60° C., 65° C., 70° C., 75° C., 80° C., 85° C., 90° C., 95° C., or 100° C.). In particular embodiments, a Cas9 protein variant disclosed herein has nuclease activity at temperatures above 70° C. (e.g., 72° C., 75° C., 77° C., 80° C., 82° C., 85° C., 87° C., 90° C., 92° C., 95° C., 97° C., or 100° C.). Assays are available to determine the cleavage activity and/or thermal stability of a Cas9 protein or a variant thereof at a specific temperature. For example, to assay cleavage activity, a Cas9 protein or a variant thereof may be incubated with the appropriate sgRNA to form a ribonucleoprotein complex. A nucleic acid containing the target DNA and the PAM sequence may be incubated with the ribonucleoprotein complex at the desired temperature (e.g., 20° C., 25° C., 30° C., 35° C., 40° C., 45° C., 50° C., 55° C., 60° C., 65° C., 70° C., 75° C., 80° C., 85° C., 90° C., 95° C., or 100° C.) for different lengths of time (e.g., between 5 minutes to 1 hour; e.g., 5 minutes, 10, minutes, 20, minutes, 30 minutes, 40, minutes, 50 minutes, or 1 hour). The cleavage reaction may be terminated by adding a protease (e.g., Proteinase K), EDTA, and/or SDS. The cleavage DNA products may be assessed by extracting the DNA products and running the DNA products on an agarose gel. The DNA products from the cleavage reaction would be separated on the agarose gel as shorter nucleotide sequences compared to the original target DNA prior to cleavage. Multiple reactions may be performed in parallel to compare the cleavage activities of different Cas9 proteins or variants thereof side by side (e.g., comparing the cleavage activities of a Cas9 protein variant disclosed herein and another Cas protein, such as GeoCas9 and ThermoCas9).

Thermal Stability of a Cas9 protein or a variant thereof as disclosed herein may also be assessed using analytical techniques, such as differential scanning calorimetry. Differential scanning calorimetry measures the molar heat capacity of reaction samples as a function of temperature. In the case of protein samples, differential scanning calorimetry profiles provide information about thermal stability, and may serve as a structural “fingerprint” that can be used to assess structural conformation. It may be performed using a differential scanning calorimeter that measures the thermal transition temperature (melting temperature; Tm) and the energy required to disrupt the interactions stabilizing the tertiary structure (enthalpy; ΔH) of proteins. Comparisons may be made between different Cas9 proteins, e.g., a wild-type Cas9 protein and a Cas9 protein variant, and differences in derived values indicate differences in thermal stability and structural conformation between the two proteins. Differential scanning calorimetry may be used to obtain a complete thermodynamic profile of the protein unfolding process. In some embodiments, a Cas9 protein variant as disclosed herein has a higher melting temperature, Tm, compared to a wild-type Cas9 protein (e.g., GeoCas9 or ThermoCas9).

### 3. Single-Guide RNA (sgRNA)

A Cas9 protein variant disclosed herein may be guided to its target DNA by a single-guide RNA (sgRNA). An sgRNA is a version of the naturally occurring two-piece guide RNA (crRNA and tracrRNA) engineered into a single, continuous sequence. An sgRNA may contain a guide sequence (e.g., the crRNA equivalent portion of the sgRNA) that targets the Cas protein to the target DNA and a scaffold sequence that interacts with the Cas protein (e.g., the tracrRNAs equivalent portion of the sgRNA).

### 3.1 Guide Sequence

The guide sequence in the sgRNA may be complementary to a specific sequence within a target DNA. The 3′ end of the target DNA sequence must be followed by a PAM sequence. Approximately 20 nucleotides upstream of the PAM sequence is the target DNA. In general, a Cas9 protein or a variant thereof cleaves about three nucleotides upstream of the PAM sequence. The guide sequence in the sgRNA can be complementary to either strand of the target DNA.

In some embodiments, the guide sequence of an sgRNA may comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end of the sgRNA that can direct the Cas protein to the target DNA site using RNA-DNA complementarity base pairing. In some embodiments, the guide sequence of an sgRNA comprises about 100 nucleic acids at the 5′ end of the sgRNA that can direct the Cas protein to the target DNA site using RNA-DNA complementarity base pairing. In some embodiments, the guide sequence comprises 20 nucleic acids at the 5′ end of the sgRNA that can direct the Cas protein to the target DNA site using RNA-DNA complementarity base pairing. In some embodiments, the guide sequence comprises at least 22 (e.g., 23, 24, 25, 26, 27, 28, 29, 30, 31, 32, 33, 34, 35, 36, 37, 38, 39, 40, 41, 42, 43, 44, 45, 46, 47, 48, 49, or 50) nucleic acids at the 5′ end of the sgRNA that can direct the Cas protein to the target DNA site using RNA-DNA complementarity base pairing. In some embodiments, the guide sequence comprises between 22 and 25 (e.g., 22, 23, 24, or 25) nucleic acids at the 5′ end of the sgRNA that can direct the Cas protein to the target DNA site using RNA-DNA complementarity base pairing. In other embodiments, the guide sequence comprises less than 20, e.g., 19, 18, 17, 16, 15 or less, nucleic acids that are complementary to the target DNA site. In some instances, the guide sequence in the sgRNA contains at least one nucleic acid mismatch in the complementarity region of the target DNA site. In some instances, the guide sequence contains about 1 to about 10 nucleic acid mismatches in the complementarity region of the target DNA site.

### 3.2 Scaffold Sequence

The scaffold sequence in the sgRNA may serve as a protein-binding sequence that interacts with the Cas protein or a variant thereof. In some embodiments, the scaffold sequence in the sgRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the sgRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

In some embodiments, the scaffold sequence in the sgRNA has at least 75% sequence identity (e.g., 75%, 80%, 85%, 90%, 91%, 92%, 93%, 94% 95%, 96%, 97%, 98%, 99%, or 100% sequence identity) to the sequence of: GUUGUGAUUUGCUUUCAAAGAAAUUUGAAGCAAAUCACAAUAAGGAUUUUUCCGUUGUGAAAACAUU UACAGUAGUCCCGAUGCAAACCAUCGGGAUUGUUGUUUU (SEQ ID NO:7). In some embodiments, the scaffold sequence in the sgRNA contains a fragment (e.g., at least 20 nucleotides; 20, 30, 40, 50, 60, 70, 80, 90, 100, or more nucleotides) of the sequence of SEQ ID NO:7. In some embodiments, the scaffold sequence in the sgRNA contains a fragment (e.g., at least 20 nucleotides; 20, 30, 40, 50, 60, 70, 80, 90, 100, or more nucleotides) of the sequence of SEQ ID NO:7 and at least 75% sequence identity (e.g., 75%, 80%, 85%, 90%, 91%, 92%, 93%, 94% 95%, 96%, 97%, 98%, 99%, or 100% sequence identity) to the sequence of SEQ ID NO:7. In particular embodiments, the scaffold sequence in the sgRNA has the sequence of SEQ ID NO:7.

### 3.3 Modified sgRNA

In particular embodiments, the sgRNA may be chemically modified. Without being bound by any particular theory, sgRNAs containing one or more chemical modifications may have increased activity, stability, and specificity and/or decreased toxicity compared to a corresponding unmodified sgRNA. Non-limiting advantages of modified sgRNAs include greater ease of delivery into target cells, increased stability, increased duration of activity, and reduced toxicity. Modified sgRNAs may provide higher frequencies of on-target genetic editing (e.g., homologous recombination), improved activity, and/or specificity compared to their unmodified sequence equivalents.

In some embodiments, one or more nucleotides of the guide sequence and/or one or more nucleotides of the scaffold sequence in the sgRNA can be a modified nucleotide. For instance, a guide sequence that is about 20 nucleotides in length may have 1 or more, e.g., 1, 2, 3, 4, 5, 6, 7, 8, 9, 10, 11, 12, 13, 14, 15, 16, 17, 18, 19, or 20 modified nucleotides. In some cases, the guide sequence includes at least 2, 3, 4, 5, 6, 7, 8, 9, 10, or more modified nucleotides. In other cases, the guide sequence includes at least 2, 3, 4, 5, 6, 7, 8, 9, 10, 11, 12, 13, 14, 15, 16, 17, 19, 20, or more modified nucleotides. The modified nucleotide can be located at any nucleic acid position of the guide sequence. In other words, the modified nucleotides can be at or near the first and/or last nucleotide of the guide sequence, and/or at any position in between. For example, for a guide sequence that is 20 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, and/or position 20 of the guide sequence. In certain instances, from about 10% to about 30%, e.g., about 10% to about 25%, about 10% to about 20%, about 10% to about 15%, about 15% to about 30%, about 20% to about 30%, or about 25% to about 30% of the guide sequence can comprise modified nucleotides. In other instances, from about 10% to about 30%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the guide sequence can comprise modified nucleotides.

In some embodiments, the scaffold sequence of the modified sgRNA contains one or more modified nucleotides. For example, a scaffold sequence that is about 100 nucleotides in length may have 1 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 modified nucleotides. In some instances, the scaffold sequence includes at least 2, 3, 4, 5, 6, 7, 8, 9, 10, or more modified nucleotides. In other instances, the scaffold sequence includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the scaffold sequence. For example, the modified nucleotides can be at or near the first and/or last nucleotide of the scaffold sequence, and/or at any position in between. For example, for a scaffold sequence that is about 100 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, position 39, position 40, position 41, position 42, position 43, position 44, position 45, position 46, position 47, position 48, position 49, position 50, position 51, position 52, position 53, position 54, position 55, position 56, position 57, position 58, position 59, position 60, position 61, position 62, position 63, position 64, position 65, position 66, position 67, position 68, position 69, position 70, position 71, position 72, position 73, position 74, position 75, position 76, position 77, position 78, position 79, position 80, position 81, position 82, position 83, position 84, position 85, position 86, position 87, position 88, position 89, position 90, position 91, position 92, position 93, position 94, position 95, position 96, position 97, position 98, position 99, and/or position 100 of the sequence. In some instances, from about 1% to about 10%, e.g., about 1% to about 8%, about 1% to about 5%, about 5% to about 10%, or about 3% to about 7% of the scaffold sequence can comprise modified nucleotides. In other instances, from about 1% to about 10%, e.g., about 1%, about 2%, about 3%, about 4%, about 5%, about 6%, about 7%, about 8%, about 9%, or about 10% of the scaffold sequence can comprise modified nucleotides.

The modified nucleotides of the sgRNA can include a modification in the ribose (e.g., sugar) group, phosphate group, nucleobase, or any combination thereof. In some embodiments, the modification in the ribose group comprises a modification at the 2′ position of the ribose. For example, the phosphodiester linkages of a native or natural RNA may be modified to include at least one of a nitrogen or sulfur heteroatom. In some backbone-modified ribonucleotides, the phosphoester group connecting to adjacent ribonucleotides may be replaced by a modified group, e.g., a phosphothioate group. In certain sugar-modified ribonucleotides, the 2′ moiety is a group selected from H, OR, R, halo, SH, SR, NH₂, NHR, NR₂ or ON, wherein R is C1-C6 alkyl, alkenyl or alkynyl and halo is F, Cl, Br or I. In some embodiments, the sugar-modified ribonucleotide comprises a 2′-O-methyl nucleotide.

It should be noted that any of the modifications described herein may be combined and incorporated in the guide sequence and/or the scaffold sequence of the modified sgRNA. In some cases, the modified sgRNAs also include a structural modification such as a stem loop, e.g., M2 stem loop or tetraloop. The chemically modified sgRNAs can be used with any CRISPR-associated or RNA-guided technology. A modified sgRNA can serve as a substrate for a Cas9 protein variant disclosed herein.

### 3.4 Tools for sgRNA Design

An sgRNA may be selected using a software. As a non-limiting example, considerations for selecting an sgRNA can include, e.g., the PAM sequence for the Cas9 protein to be used, and strategies for minimizing off-target modifications. Tools, such as NUPACK® and the CRISPR Design Tool, can provide sequences for preparing the sgRNA, for assessing target modification efficiency, and/or assessing cleavage at off-target sites.

The following guidelines may be followed as an example of selecting a target DNA and designing sgRNA. First, to select a target DNA, the 3′ end of the target DNA sequence must be followed by a PAM sequence. Approximately 20 nucleotides upstream of the PAM sequence is the target DNA. In general, a Cas9 protein or a variant thereof cleaves about three nucleotides upstream of the PAM sequence. The PAM sequence is required for target DNA cleavage, but it is not part of the sgRNA and therefore should not be included in the sgRNA. The guide sequence in the sgRNA can be complementary to either strand of the target DNA. As described further herein, an sgRNA for a Cas9 protein variant disclosed herein may be designed based on computational predictions using crRNAs and tracrRNAs of other type II-C Cas proteins.

As described in Example 1, the sequence one suitable sgRNA is: [GGGAAUAGUUACAUUACUAUCUGUA]GUUGUGAUUUGCUUUCAAAGAAAUUUGAAGCAAAUCACAA UAAGGAUUUUUCCGUUGUGAAAACAUUUACAGUAGUCCCGAUGCAAACCAUCGGGAUUGUUGUUUU (SEQ ID NO:9), where the guide sequence is in brackets and the scaffold sequence (SEQ ID NO:7) is in bold. The sequence of the 100-bp DNA target template used in the experiment is: CATGGTCAGACAAGCTTACTAGTAAAGGATCCACGGGTACCGAGCTTCCATCC[GGGAATAGTTACATTACTAT CTGTA]GGACATGAAAGAATTCGTAAT (SEQ ID NO:8), where the target DNA region is in brackets and the PAM sequence (which falls within the PAM motif NVRNAT (SEQ ID NO:6), where N is any nucleotide, V is A, G or C, and R is G or A, or the PAM motif NRRNAT (SEQ ID NO:13), where N is any nucleotide and R is G or A) is in bold.

### 4. Expression Systems

Methods for introducing proteins and nucleic acids into a cell are known in the art. Any known method can be used to introduce a protein or a nucleic acid (e.g., a Cas9 protein, an RNA, or a nucleic acid or vector encoding a Cas9 protein or associated RNA) into a cell, e.g., a mammalian cell (e.g., a human cell). Non-limiting examples of suitable methods for introducing IgnaviCas9 into a bacterial or eukaryotic cell include electroporation (e.g., nucleofection), viral or bacteriophage infection, transfection, conjugation, protoplast fusion, and the like.

For sgRNA expression and delivery, in some embodiments, a nucleotide sequence encoding the sgRNA is cloned into an expression cassette or an expression vector. In certain embodiments, the nucleotide sequence is produced by PCR and contained in an expression cassette. For instance, the nucleotide sequence encoding the sgRNA can be PCR amplified and appended to a promoter sequence, e.g., a U6 RNA polymerase III promoter sequence. In other embodiments, the nucleotide sequence encoding the sgRNA is cloned into an expression vector that contains a promoter, e.g., a U6 RNA polymerase III promoter, and a transcriptional control element, enhancer, U6 termination sequence, one or more nuclear localization signals, etc. In some embodiments, the expression vector is multicistronic or bicistronic and can also include a nucleotide sequence encoding a fluorescent protein, an epitope tag and/or an antibiotic resistance marker. In other embodiments, the sgRNA may be chemically synthesized. The sgRNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J. American Chemical Society 125, 940-950 (2003).

Suitable expression vectors for expressing the sgRNA are commercially available from sources such as Addgene, Sigma-Aldrich, and Life Technologies. Non-limiting examples of other expression vectors include pX330, pSpCas9, pSpCas9n, pSpCas9-2A-Puro, pSpCas9-2A-GFP, pSpCas9n-2A-Puro, the GeneArt® CRISPR Nuclease OFP vector, the GeneArt® CRISPR Nuclease OFP vector, and the like.

### 5. Applications

IgnaviCas9 and IgnaviCas9 ribonucleoprotein complex described herein may be used for any purpose or method for which CRISPR-Cas9 type II system are suitable. The wide active temperature range of the Cas9 protein variants described herein is a unique property than can be harnessed for a host of molecular biology applications. In particular, the high thermal stability of the Cas9 protein variants described herein enables the proteins to be used in environments and applications requiring elevated temperatures (e.g., at least 70° C. or higher), where other proteins may be inactive (e.g., GeoCas9 and ThermoCas9).

### 5.1 Removing Unwanted Species in Sequencing

The advancement of a large variety of next-generation sequencing technologies (see, e.g., Levy and Myers, Annual Review of Genomics and Human Genetics 17:95-115, 2016) has generated a need for a broadly applicable method to remove, prior to sequencing, unwanted high-abundance species that are generated during amplification (e.g., during preparation of sequencing libraries). See, for example, Gu et al., Genome Biology 17:41, 2016, Ramani and Shendure, Genome Biology 17:42, 2016, and Hardigan et al., BioRxiv, May 2018). Given that amplification reactions, e.g., PCR, are generally performed through cycles of high temperatures (e.g., annealing temperature between 48° C. and 72° C., extension temperature between 68° C. and 72° C., and denaturation temperature between 92° C. and 98° C.), the highly thermostable Cas9 protein variants disclosed herein are particularly suited for simultaneous use in the amplification reactions. In some embodiments, the Cas9 protein variants disclosed herein complexed with one or more sgRNAs may be added into the amplification reactions to remove unwanted species during the generation of sequencing libraries, thus preventing them from consuming sequencing space. The one or more sgRNAs may be designed to target one or more unwanted species in the libraries for cleavage.

The activity of IgNAviCas9 at both moderate and high temperatures led to the consideration of how IgnaviCas9 could be integrated into polymerase chain reactions (PCRs) to eliminate primer-dimers. Formed through hybridization and subsequent amplification of primers with complementary bases, primer-dimers compete with amplification of the desired DNA target, reducing the efficiency of PCR. This issue is particularly prevalent in multiplexed PCR and limits the number of loci that can be concurrently amplified. Including IgnaviCas9 with sgRNA targeting the predicted primer-dimer(s) in a given PCR can reduce their formation and reduce their proportion of final products in a PCR. As demonstrated herein, IgnaviCas9 can be leveraged to remove 16s ribosomal rRNA (rRNA) from bacterial RNA-Seq libraries as they are amplified during library preparation, underscoring the benefits provided by the protein's thermostability in improving molecular biology and genomic workflows.

### 5.2 In Vivo Use

The exceptional thermostability of IgnaviCas9 is also a feature that makes the protein well suited for in vivo use. In particular, increased stability suggests that IgnaviCas9 may have a longer lifetime in plasma than those of canonical variants and thus, may be more effective for applications such as gene therapies (Long) or lineage tracing in complex organisms (Schmidt). While organisms dwelling at higher temperatures are typically simple microbes, these microbes can catalyze industrial processes like fermentation. The improved ability to further engineer these thermophilic bacteria by means of IgnaviCas9 may facilitate the development and broader implementation of these processes.

### 6. Examples

### 6.1 Example 1: Mini-Metagenomic Identification and Phylogenetic Characterization

This example describes mini-metagenomic identification, phylogenetic characterization, expression, and purification of IgnaviCas9.

Microfluidic mini-metagenomic sequencing of a hot spring sample from the Mound Spring of Lower Geyser Basin of Yellowstone National Park (permit YELL-2009-SCI-5788) yielded a full CRISPR array from a new bacterium in the Ignavibacteriae phylum. This genome comprised of a single 3.4 Mb contig representing a novel lineage in the Ignavibacteriae phylum. The temperature of the sample was recorded as 55° C. and that of the hot spring as >90° C.

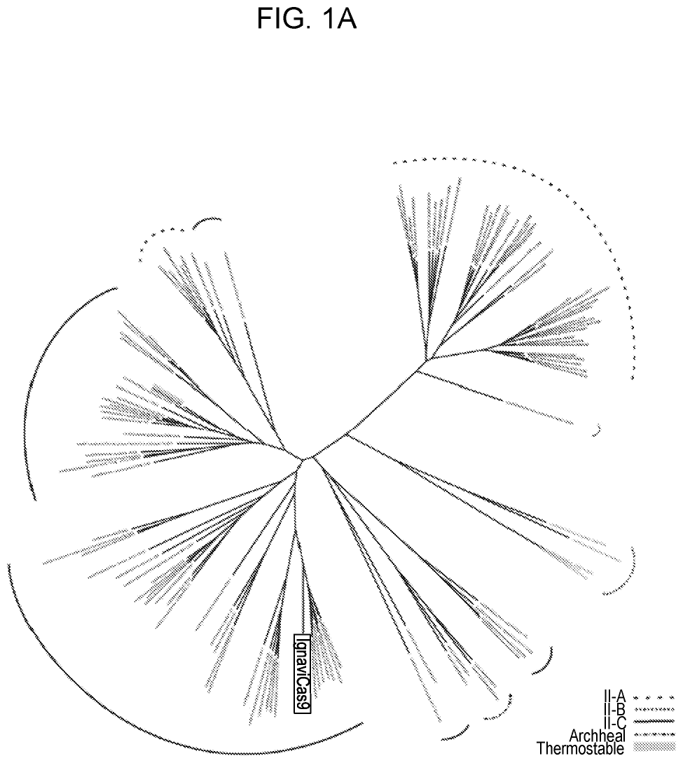

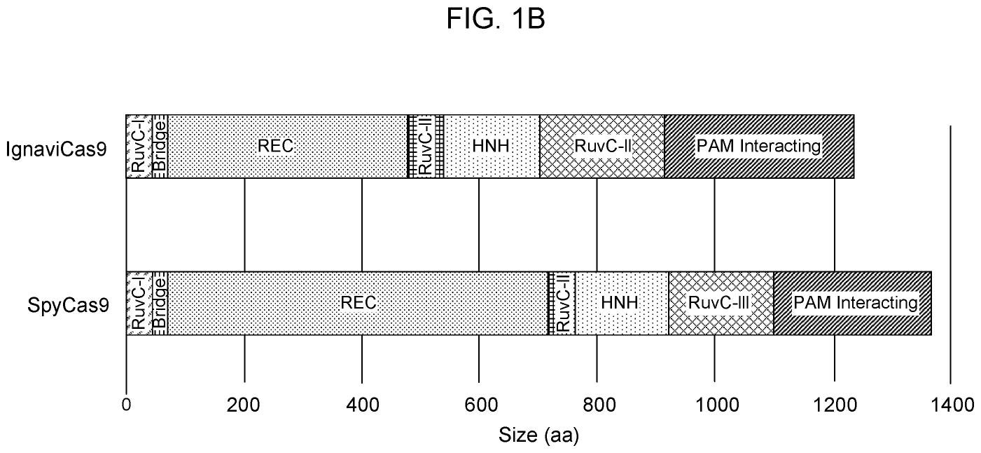

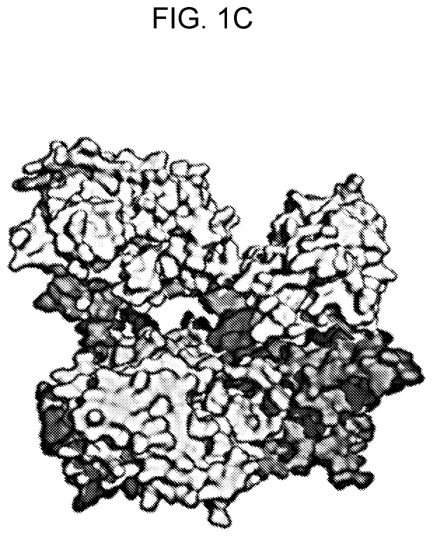

The isolated CRISPR array contained a Cas9 protein, Cas1 protein, and Cast protein along with 38 unique spacers. The absence of a Csn2 and Cas4 protein suggested that the Ignavibacterium possessed a type II-C system (Mir), which was confirmed by phylogenetic comparison of IgnaviCas9 to other type II Cas9 proteins (FIG. 1A). Briefly, multiple sequence alignment of amino acid sequences of representative type II Cas9 proteins was performed using MAFFT (Katoh), and a maximum-likelihood phylogenetic tree was constructed using RA×ML with the PROTGAMMALG substitution model and 100 bootstrap samplings (Stamatakis). IgnaviCas9 ended up within the type II-C portion of the resulting tree, and the in vitro validated type II-C Cas9 to which it is most similar is that of Parvibaculum lavamentivorans (Ran), a mesophilic bacterium with an optimal growth temperature of 30° C.

### 6.2 Example 2: Expression and Purification

At 1240 amino acids long, IgnaviCas9 is shorter than SpyCas9 (1368 amino acids) but longer than ThermoCas9 (1082 amino acids) or GeoCas9 (1087 amino acids). Through homology modeling and sequence alignment, the smaller size of IgnaviCas9 compared to SpyCas9 was found to arise from its reduced REC lobe (FIG. 1B), which is consistent with other smaller Cas9s (Ran). While IgnaviCas9 is larger than other in vitro validated type II-C Cas9 proteins, that IgnaviCas9 is shorter than SpyCas9 is an advantage for applications involving its delivery via adeno-associated viruses (Wu). The nucleic acid sequence of IgnaviCas9 (SEQ ID NO:2) was E. coli codon-optimized to produce nucleic acid sequence of SEQ ID NO:3, which was cloned into a Cas9-expression vector, a pET-based vector with an N-terminal hexahistidine, maltose binding protein, and tobacco etch virus sequence and C-terminal nuclear localization sequences. BL21 E. coli cells were transformed with this plasmid and cultured to express IgnaviCas9. After cultures reached an OD₆₀₀ nm of 0.5, expression was induced by adding IPTG to give a final concentration of 0.5 mM. The cultures were allowed to incubate for 7 hours at 16° C. Cells were harvested via centrifugation, and IgnaviCas9 was purified using ion exchange and size exclusion chromatography per previously described methods (Gu). IgnaviCas9-containing fractions were pooled, supplemented with glycerol to a final concentration of 50%, and stored at −80° C. until used. The purification provided 12 mg of IgnaviCas9 from 4 L of culture for downstream experiments.

### 6.3 Example 3: Engineering IgnaviCas9 sgRNA

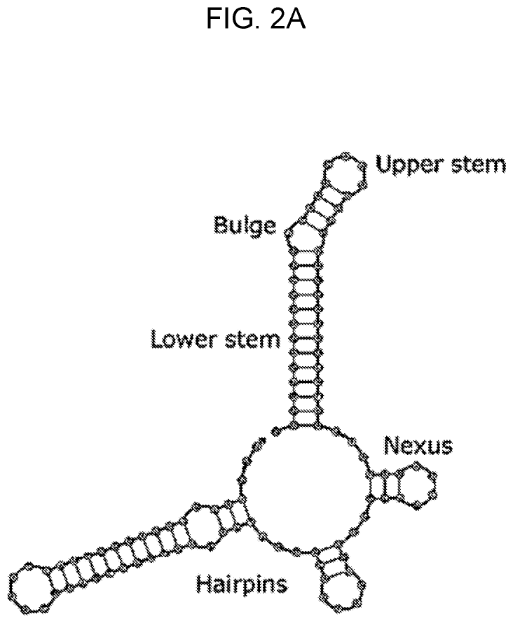

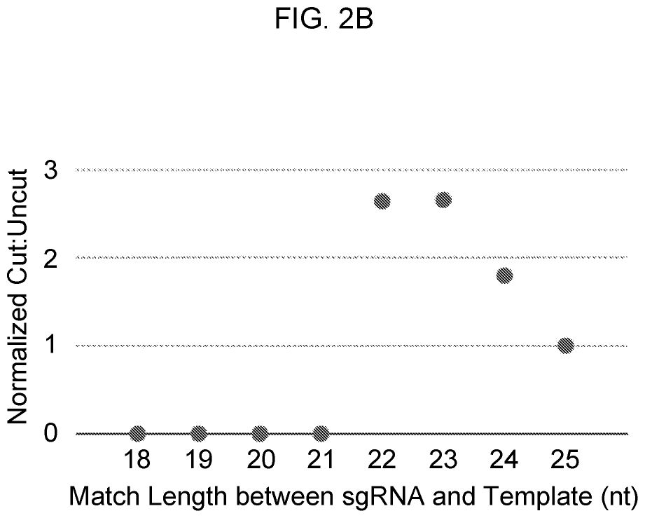

IgnaviCas9 falls within the type II-C classification and its sgRNA was designed based on computational prediction of its crRNA and tracrRNA from the available CRISPR array sequence. The crRNA and tracrRNA were identified from the IgnaviCas9 CRISPR locus by searching for complementarity between candidate sequences that allowed for the formation of the requisite features when linked by a 5′-GAAA-3′ tetraloop (Briner). Possible sgRNA sequences were tested through secondary structure prediction using NUPACK (Zadeh). Combinations of potential crRNA and tracrRNA sequences that together allowed for the formation of the lower stem, bulge, upper stem, nexus, and hairpin features were searched (FIG. 2A). RNA secondary structure prediction of the designed sgRNA showed that all desired features remained present at temperatures of 60° C. for default NUPACK program settings, underscoring the potential of IgnaviCas9 to cleave DNA at temperatures outside of the mesophilic range. DNA corresponding to the sgRNA including the target of interest was placed under control of a T7 promoter and synthesized (Integrated DNA Technologies). sgRNAs were transcribed using the MEGAshortScript T7 Transcription Kit (Thermo Fisher Scientific) with overnight incubation and purified using the MEGAclear Transcription Clean-Up Kit (Thermo Fisher Scientific). The sgRNA sequence preceded by 25 nucleotides of spacer sequence was transcribed for use in preliminary experiments.

### 6.4 Example 4: IgnaviCas9 PAM Determination and sgRNA-Spacer Match Length Refinement

The protospacer adjacent motif (PAM), the sequence directly downstream of a nucleic acid target cleavable by CRISPR systems, varies between different species and prevents the host genome from being attacked (Mojica). As an initial approach, a double-stranded linear DNA containing a spacer sequence followed by a PAM from an in vitro validated type II-C CRISPR system was designed. Cleavage assays were performed by incubating the assorted DNA substrates with a ribonucleoprotein complex (RNP) of IgnaviCas9 and sgRNA targeting the spacer sequence as described below.

The purified IgnaviCas9 and transcribed sgRNA were used to cleave DNA targets at desired temperatures. The sequence of the sgRNA is: [GGGAAUAGUUACAUUACUAUCUGUA]GUUGUGAUUUGCUUUCAAAGAAAUUUGAAGCAAAUCACAA UAAGGAUUUUUCCGUUGUGAAAACAUUUACAGUAGUCCCGAUGCAAACCAUCGGGAUUGUUGUUUU (SEQ ID NO:9), where the guide sequence is in brackets and the scaffold sequence (SEQ ID NO:7) is in bold. DNA target templates approximately 100 bp long used in the PAM determination experiments and short-length temperature range testing were synthesized (Integrated DNA Technologies). The sequence of the 100-bp DNA target template is: CATGGTCAGACAAGCTTACTAGTAAAGGATCCACGGGTACCGAGCTTCCATCC[GGGAATAGTTACATTACTAT CTGTA]GGACATGAAAGAATTCGTAAT (SEQ ID NO:8), where the target DNA region is in brackets and the PAM sequence (which falls within the PAM motif NVRNAT (SEQ ID NO:6), where N is any nucleotide, V is A, G or C, and R is G or A, or the PAM motif NRRNAT (SEQ ID NO:13), where N is any nucleotide and R is G or A) is in bold. Plasmid templates were generated by linearizing the pwtCas9 plasmid (Qi) using Xhol (New England Biolabs).

IgnaviCas9 and the appropriate sgRNA were incubated together in reaction buffer at 37° C. for 10 minutes before adding the DNA target added to the reaction. The reaction was then incubated at the specified temperature for 30 minutes. The final composition of each reaction was 5 nM substrate DNA, 100 nM IgnaviCas9, 150 nM sgRNA, 20 mM Tris-HCl pH 7.6, 100 mM KCl, 5 mM MgCl2, 1 mM DTT, and 5% glycerol (volume per volume).

Each reaction was quenched using 6× Quench Buffer (15% glycerol, 100 mM EDTA) and then underwent Proteinase K digestion at room temperature for 20 minutes before being loaded into a chip for fragment analysis using the Bioanalyzer (Agilent).

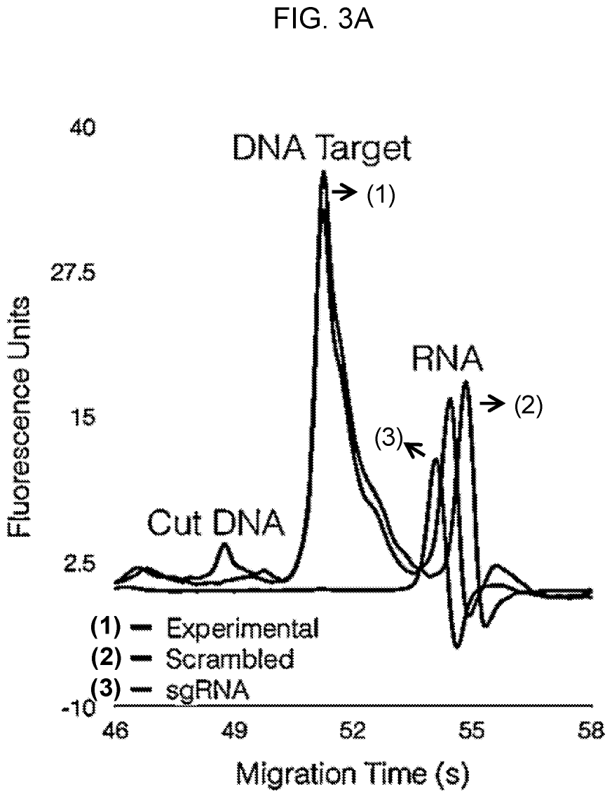

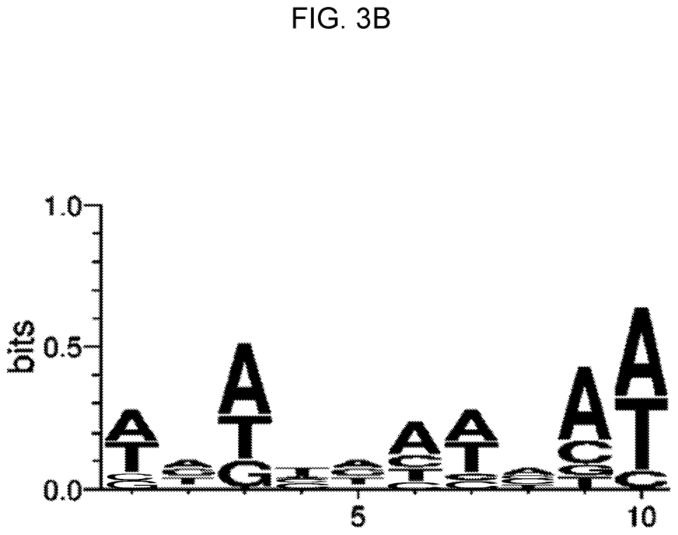

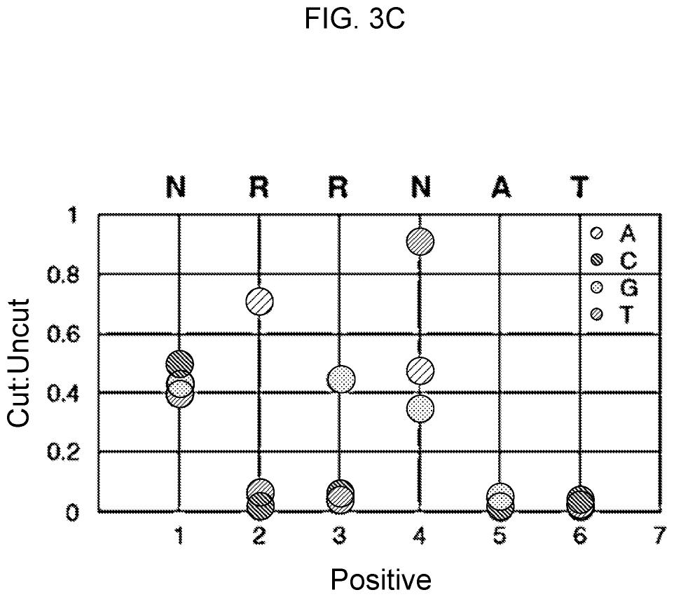

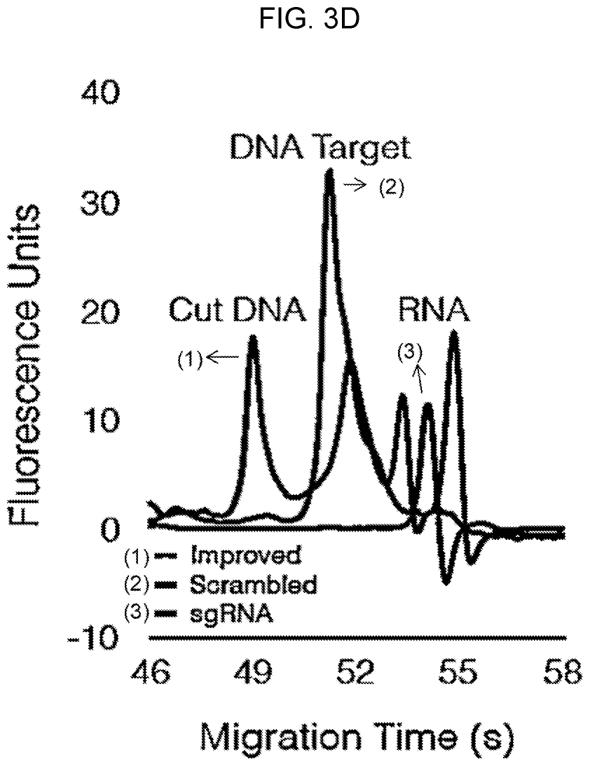

It was found that IgnaviCas9 cleaved the DNA substrate with the PAM sequence CCACATCGAA (SEQ ID NO:4), containing the NNNCAT motif from P. lavamentivorans (FIG. 3A). A control reaction was used as a point of reference and differed in that the sgRNA included contained a scrambled version of the spacer. That the DNA substrate with the PAM from P. lavamentivorans was cleaved was an exciting result, given that the P. lavamentivorans Cas9 is the homolog to which IgnaviCas9 is most similar per the earlier phylogenetic analysis.

The 38 spacers found in the IgnaviCas9 CRISPR array were used to isolate possible protospacers from the environmental sample in which IgnaviCas9 was found. By using BLAST to search the environmental sequences, 10 bp sequences flanking the spacer that were different from the repeat sequence by an edit distance of at least 5 were collected. The sequence logo created using unique sequences meeting these criteria suggested that the PAM was likely to be adenine-rich (FIG. 3B). Subsequently, a new DNA substrate was designed by modifying the aforementioned DNA substrate that was cut by IgnaviCas9 to include AGACATGAAA (SEQ ID NO:5), an adenine-rich version of the P. lavamentivorans PAM. This choice was also informed by the results of a randomer depletion experiment. Briefly, template containing a 10-bp long randomer was used as the DNA substrate in a cleavage reaction. The resulting mixture of fragments underwent sequencing, and a sequence logo was generated using randomers depleted relative to their presence in the starting library. In a cleavage reaction performed as before, IgnaviCas9 was able to better cleave the DNA substrate containing the refined PAM (FIG. 3D). IgnaviCas9 cleaved the new DNA substrate in a cleavage reaction performed as before.

The PAM recognized by IgnaviCas9 was finalized by testing DNA substrates containing the aforementioned adenine-rich P. lavamentivorans PAM with single nucleotide substitutions at each of the 10 positions directly downstream of the spacer (FIG. 3C). Disruption of IgnaviCas9 cleavage by a particular substitution demonstrated that the position of the substitution was important to the PAM and that the nucleotide was not part of the PAM. It was found that NVRNAT (SEQ ID NO:6, wherein N is any nucleotide, V is A, G or C, and R is G or A) or NRRNAT (SEQ ID NO:13, wherein N is any nucleotide and R is G or A) is the PAM motif recognized by IgnaviCas9; all substitutions at positions past the sixth bp downstream of the spacer sequence were tolerated (FIG. 3C). In some embodiments, the Cas9 protein variant disclosed herein recognizes the PAM sequence GGACAT (SEQ ID NO:10), which falls within the PAM motif NVRNAT (SEQ ID NO:6) or NRRNAT (SEQ ID NO:13).

Having established IgnaviCas9s PAM, the length of spacer included in the sgRNA was varied to determine which lengths were optimal. It was demonstrated that IgnaviCas9 cleaves DNA when the sgRNA includes spacer lengths of 22 to 25 nucleotides, with a slight improvement in performance with 22 or 23 nucleotides spacer lengths (FIG. 2B). Cleavage does not occur for sgRNA with shorter spacer lengths. The spacer sizes IgnaviCas9 prefers overlap with those favored by ThermoCas9 (19 to 25 nucleotides) and GeoCas9 (21 or 22 nucleotides) but are slightly larger than the 20 nucleotides spacer length typically used with SpyCas9.

### 6.5 Example 5: Active Temperature Range Assessment

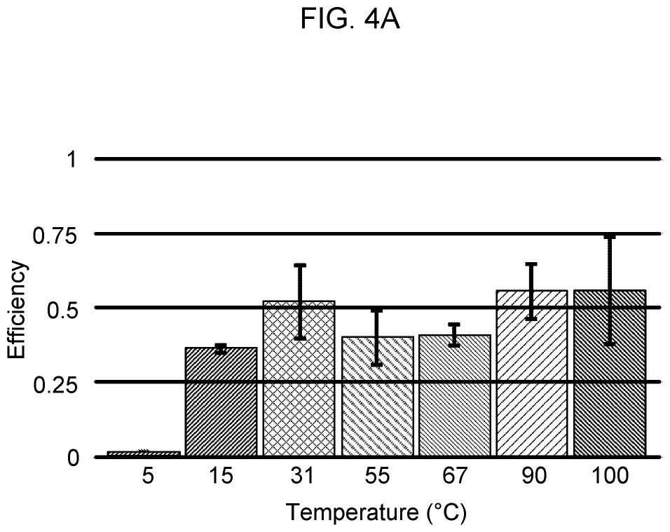

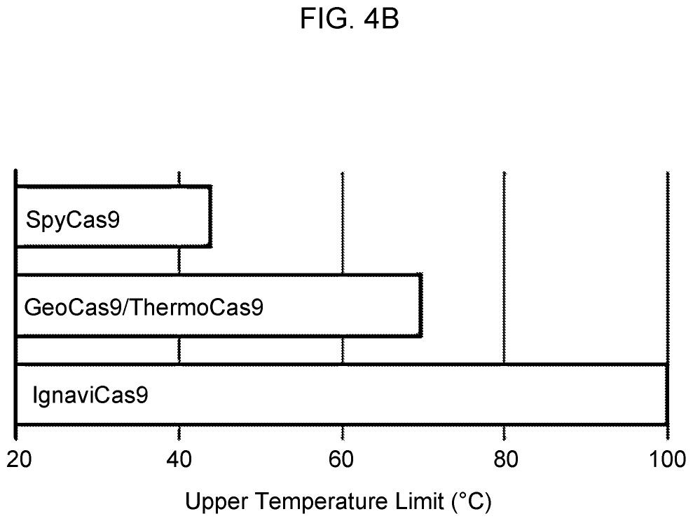

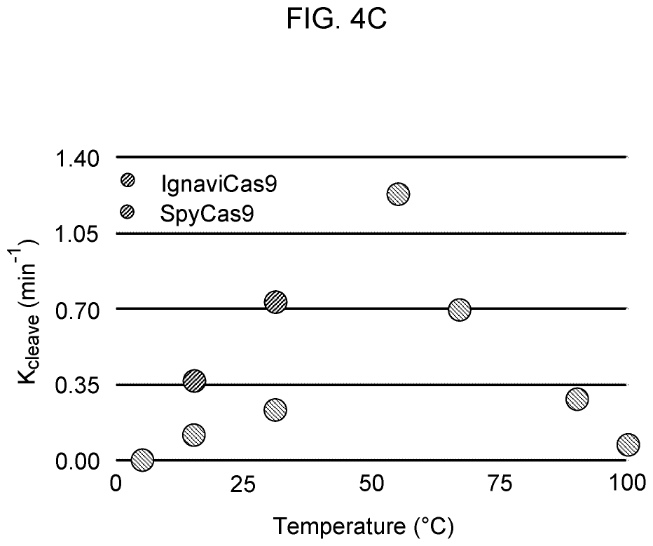

Through the PAM determination experiments conducted at 52° C., it was confirmed that IgnaviCas9 has nuclease activity at temperatures above those of the active range of SpyCas9, which has been reported as between 20° C. and 44° C. (Mougiakos et al., Nature Communications 8(1):1647, 2017 and Wiktor et al., Nucleic acids research 44(8):3801-10, 2016). The temperature range over which IgnaviCas9 has nuclease activity was characterized by performing cleavage assays between 5° C. and 100° C. (FIG. 4A). It was found that its performance in cutting various DNA targets, including longer templates like plasmid DNA (FIG. 4A), extended across the entire range tested, which reaches beyond the upper active temperature limit of other thermostable Cas9 proteins (FIG. 4B). That IgnaviCas9 remains active at high temperatures and across a wide thermal range (FIG. 4C) suggests that it is particularly stable and likely more specific in its targeting than SpyCas9, given the lower mismatch tolerance of other thermostable Cas9 proteins compared to SpyCas9 (Harrington et al., Nature Communications 8(1):1424, 2017 and Mougiakos et al., Nature Communications 8(1):1647, 2017). Like ThermoCas9 (Mougiakos et al., Nature Communications 8(1):1647 (2017)), its spacer-protospacer mismatch tolerance does increase with temperature. More generally, IgnaviCas9 is more sensitive to mismatches proximal to the PAM than those distal, which is consistent with the behavior of other Cas9 proteins.

### 6.6 Example 6: Removal of Undesired Amplicons

This example describes using IgnaviCas9 to remove undesired amplicons.

In particular, the activity of IgnaviCas9 at both moderate and high temperatures led to the consideration of how IgnaviCas9 could be integrated into molecular biology and genomic workflows to eliminate undesired amplicons. IgnaviCas9 could be leveraged to reduce the presence of 16s rRNA in bacterial libraries for RNA-Sequencing. By limiting the amplification of cDNA derived from 16s rRNA during library preparation, libraries that contain more information about the expression profiles of interest from the bacterial cells sampled could be created. See Gu et al. Depletion of Abundant Sequences by Hybridization (DASH): using Cas9 to remove unwanted high-abundance species in sequencing libraries and molecular counting applications. Genome Biology. 2016 December; 17(1):41.

When performing RNA-seq of actively growing bacterial strains or generating meta-transcriptomic data from environmental samples, reads from 16s rRNA genes are typically highly abundant and reduce sequencing bandwidth of expression profiles of interest. IgnaviCas9 was deployed during the PCR step of the sequencing library preparation workflow to cleave library fragments derived from 16s rRNA, thus reducing their presence in the final library without adding steps to the workflow. Previous work using mesophilic Cas9 in an additional workflow step prior to amplification has shown that this general idea has powerful applications (Gu et al. Genome Biology. 2016 December; 17(1):41), and it is demonstrated that targeted depletion with IgnaviCas9 can be achieved during amplification, thus offering a more streamlined workflow and without the additional clean-up step required by existing methods.

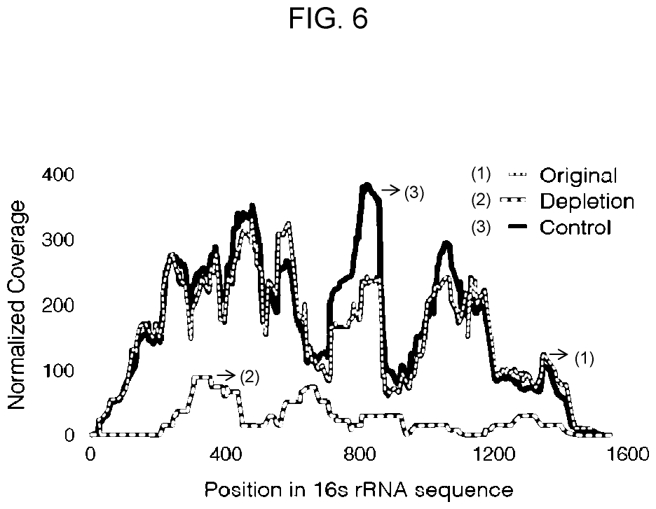

To this end, sgRNA that would target highly conserved regions in cDNA resulting from 16s rRNA was designed. IgnaviCas9 complexed to these sgRNAs was added in the combined reverse transcription and polymerase chain reaction (PCR) step of the RNA-Seq library preparation workflow. Through sequencing, it was demonstrated that simultaneous IgnaviCas9 targeting reduced the contribution of cDNA derived from 16s rRNA in the final libraries, thus enriching the portion containing transcripts of interest (FIG. 6). More broadly, the approach could be used to eliminate other unwanted amplicons, e.g., primer-dimers, as they are generated. Such implementations of IgnaviCas9 underscore its utility in improving widely used existing techniques in genomics and molecular biology.

### 6.7 Example 7: Methods

IgnaviCas9 identification, expression, and purification. IgnaviCas9 was found through mini-metagenomic sequencing of a sediment sample taken from Mound Spring in the Lower Geyser Basin area of Yellowstone National Park under permit YELL-2009-SCI-5788. The sample was placed in 50% ethanol in a 2 mL tube without any filtering and kept frozen until returning from Yellowstone to Stanford University, at which time tubes containing the samples were transferred to −80° C. for long term storage.

To compare IgnaviCas9 to other Cas9s (Burstein et al., Nature. 542, 237-241 (2017)), multiple sequence alignment of type II Cas9s was performed using MAFFT (Katoh et al., Mol. Biol. Evol. 30, 772-780 (2013)), and a maximum-likelihood phylogenetic tree was constructed using RA×ML with the PROTGAMMALG substitution model and 100 bootstrap samplings (Stamatakis, Bioinformatics. 30, 1312-1313 (2014)).

Its DNA sequence was codon-optimized for expression in E. coli and then synthesized (Integrated DNA Technologies). The resulting DNA was cloned into a pET-based vector with an N-terminal hexahistidine, maltose binding protein, and tobacco etch virus sequence and C-terminal nuclear localization sequences.

IgnaviCas9 was expressed in BL21 strain E. coli (Agilent). After cultures reached an OD600 nm of 0.5, expression was induced by adding IPTG to give a final concentration of 0.5 mM. The cultures were allowed to incubate for 7 hours at 16° C. Cells were harvested via centrifugation, and IgnaviCas9 was purified using ion exchange and size exclusion chromatography per previously described methods (Gu et al., Genome Biol. 17, 41 (2016)). IgnaviCas9-containing fractions were pooled, supplemented with glycerol to a final concentration of 50%, and stored at −80° C. until used.

sgRNA design and transcription. The crRNA and tracrRNA were identified from the IgnaviCas9 CRISPR locus by searching for complementarity between candidate sequences that allowed for the formation of the requisite features when linked by a 5′-GAAA-3′ tetraloop (Briner et al., Cold Spring Harb. Protoc. 2016, pdb-rot086785 (2016)). Possible sgRNA sequences were tested through secondary structure prediction using NUPACK (Zadeh et al., J. Comput. Chem. 32, 170-173 (2011)).

DNA corresponding to the sgRNA including the target of interest was placed under control of a T7 promoter and synthesized (Integrated DNA Technologies). sgRNAs were transcribed using the MEGAshortScript T7 Transcription Kit (Thermo Fisher Scientific) with overnight incubation and purified using the MEGAclear Transcription Clean-Up Kit (Thermo Fisher Scientific).

In vitro cleavage assays. The purified IgnaviCas9 and transcribed sgRNA were used to cleave DNA targets at desired temperatures. Templates approximately 100 bp long used in the PAM determination experiments and temperature range testing were synthesized (Integrated DNA Technologies). Plasmid templates for additional temperature range testing were generated by linearizing the pwtCas9 plasmid (Qi et al., Cell. 152, 1173-1183 (2013)) using Xhol (New England Biolabs).

IgnaviCas9 and the appropriate sgRNA were incubated together in reaction buffer at 37° C. for 10 minutes before adding the DNA target to the reaction. The reaction was immediately transferred to a thermocycler preset at the specified temperature and incubated for 30 minutes. The final composition of each reaction was 5 nM substrate DNA, 100 nM IgnaviCas9, 150 nM sgRNA, 20 mM Tris-HCl pH 7.6, 100 mM KCl, 5 mM MgCl2, 1 mM DTT, and 5% glycerol (volume per volume).

Each reaction was quenched using 6× Quench Buffer (15% glycerol, 100 mM EDTA) and then underwent Proteinase K digestion at room temperature for 20 minutes before being loaded into a chip for fragment analysis using the Bioanalyzer (Agilent). The library resulting from the PAM depletion experiment in which template containing 10-bp randomer was targeted underwent sequencing via NextSeq 500. Kinetic constants were calculated from timecourse activity data using Prism (GraphPad Software) with a one-phase exponential decay model per previously described methods (Harrington et al., Nat. Commun. 8, 1424 (2017); Strutt et al., eLife. 7, e32724 (2018)).

16s rRNA depletion in bacterial RNA-Seq libraries. Four different sgRNAs were designed to target cDNA arising from 16s rRNA sequences. The sgRNA complexed with IgnaviCas9 as described above was added to cDNA derived from E. coli RNA that underwent reverse transcription and amplification using the ScriptSeq Complete Gold Kit for Epidemiology (Epicentre).

The HiFi HotStart ReadyMixPCR Mix (KAPA) was used for the combined amplification and targeted depletion reaction, comprised of 25 μL HiFi HotStart ReadyMixPCR Mix, 1 μL ScriptSeq Index PCR Primer (Epicentre), 1 μL Reverse PCR Primer (Epicentre), 1 ng of cDNA library, 2.5 μL of 5.5 μM IgnaviCas9, 15 μL of 1400 nM sgRNA, 5 μL of IgnaviCas9 reaction buffer, and water to a total volume of 50 μL. The control reaction included 25 μL HiFi HotStart ReadyMixPCR Mix, 1 μL ScriptSeq Index PCR Primer (Epicentre), 1 μL Reverse PCR Primer (Epicentre), 1 ng of cDNA library, 2.2 μL of 6.2 μM SpyCas9 (NEB), 4.9 μL of 4200 nM SpyCas9 sgRNA, 2.5 μL of Buffer 3.1 (NEB), and water to a total volume of 50 μL. The cycling protocol used was as follows: 95° C. for 3 minutes, 30 cycles of 98° C. for 20 seconds and 75° C. for 30 seconds, and 72° C. for 1 minute.

A MiSeq Micro run was performed to sequence the original library and the test reaction that underwent concurrent amplification and targeted depletion. Resulting sequence reads were quality-filtered and trimmed using bbduk, aligned to the 16s rRNA sequence using bowtie2, and then sorted and indexed using samtools. Positional sequence coverage was determined using bedtools and subsequently compared between samples by normalizing to the average whole genome coverage in each sample.

### 7. References

Briner A E, Henriksen E D, Barrangou R. Prediction and validation of native and engineered Cas9 guide sequences. Cold Spring Harbor Protocols. 2016 Jul. 1; 2016(7):pdb-rot086785.
Cong L, Ran F A, Cox D, Lin S, Barretto R, Habib N, Hsu P D, Wu X, Jiang W, Marraffini L, Zhang F. Multiplex genome engineering using CRISPR/Cas systems. Science. 2013 Jan. 3:1231143.
Burstein D, Harrington L B, Strutt S C, Probst A J, Anantharaman K, Thomas B C, Doudna J A, Banfield J F. New CRISPR-Cas systems from uncultivated microbes. Nature. 2017 February; 542(7640):237.
Gu W, Crawford E D, O'Donovan B D, Wilson M R, Chow E D, Retallack H, DeRisi J L. Depletion of Abundant Sequences by Hybridization (DASH): using Cas9 to remove unwanted high-abundance species in sequencing libraries and molecular counting applications. Genome Biology. 2016 December; 17(1):41.
Harrington L B, Paez-Espino D, Staahl B T, Chen J S, Ma E, Kyrpides N C, Doudna J A. A thermostable Cas9 with increased lifetime in human plasma. Nature Communications. 2017 Nov. 10; 8(1):1424.
Jinek M, Chylinski K, Fonfara I, Hauer M, Doudna J A, Charpentier E. A programmable dual-RNA-guided DNA endonuclease in adaptive bacterial immunity. Science. 2012 Jun. 28:1225829.
Katoh K, Standley D M. MAFFT multiple sequence alignment software version 7: improvements in performance and usability. Molecular biology and evolution. 2013 Jan. 16; 30(4):772-80.
Kelley L A, Mezulis S, Yates C M, Wass M N, Sternberg M J. The Phyre2 web portal for protein modeling, prediction and analysis. Nature protocols. 2015 June; 10(6):845.
Koonin E V, Makarova K S, Zhang F. Diversity, classification and evolution of CRISPR-Cas systems. Current opinion in microbiology. 2017 Jun. 1; 37:67-78.
Long C, McAnally J R, Shelton J M, Mireault A A, Bassel-Duby R, Olson E N. Prevention of muscular dystrophy in mice by CRISPR/Cas9-mediated editing of germline DNA. Science. 2014 Sep. 5; 345(6201):1184-8.
Mir A, Edraki A, Lee J, Sontheimer E J. Type II-C CRISPR-Cas9 Biology, Mechanism, and Application. ACS chemical biology. 2017 Dec. 20; 13(2):357-65.
Mojica F J, Díez-Villaseñor C, García-Martínez J, Almendros C. Short motif sequences determine the targets of the prokaryotic CRISPR defence system. Microbiology. 2009 Mar. 1; 155(3):733-40.
Mougiakos I, Mohanraju P, Bosma E F, Vrouwe V, Bou M F, Naduthodi M I, Gussak A, Brinkman R B, Kranenburg R, Oost J. Characterizing a thermostable Cas9 for bacterial genome editing and silencing. Nature Communications. 2017 Nov. 21; 8(1):1647.
Mougiakos I, Bosma E F, Weenink K, Vossen E, Goijvaerts K, van der Oost J, van Kranenburg R. Efficient genome editing of a facultative thermophile using mesophilic spCas9. ACS synthetic biology. 2017 Feb. 16; 6(5):849-61.
Qi L S, Larson M H, Gilbert L A, Doudna J A, Weissman J S, Arkin A P, Lim W A. Repurposing CRISPR as an RNA-guided platform for sequence-specific control of gene expression. Cell. 2013 Feb. 28; 152(5):1173-83.
Ran F A, Cong L, Yan W X, Scott D A, Gootenberg J S, Kriz A J, Zetsche B, Shalem O, Wu X, Makarova K S, Koonin E V. In vivo genome editing using Staphylococcus aureus Cas9. Nature. 2015 April; 520(7546):186.
Schmidt S T, Zimmerman S M, Wang J, Kim S K, Quake S R. Quantitative analysis of synthetic cell lineage tracing using nuclease barcoding. ACS synthetic biology. 2017 Mar. 10; 6(6):936-42.
Stamatakis A. RA×ML version 8: a tool for phylogenetic analysis and post-analysis of large phylogenies. Bioinformatics. 2014 May 1; 30(9):1312-3.
Wang H, La Russa M, Qi L S. CRISPR/Cas9 in genome editing and beyond. Annual review of biochemistry. 2016 Jun. 2; 85:227-64.
Wiktor J, Lesterlin C, Sherratt D J, Dekker C. CRISPR-mediated control of the bacterial initiation of replication. Nucleic acids research. 2016 Apr. 1; 44(8):3801-10.
Wu Z, Yang H, Colosi P. Effect of genome size on AAV vector packaging. Molecular Therapy. 2010 Jan. 1; 18(1):80-6.
Yu F B, Blainey P C, Schulz F, Woyke T, Horowitz M A, Quake S R. Microfluidic-based mini-metagenomics enables discovery of novel microbial lineages from complex environmental samples. Elife. 2017 Jul. 5; 6:e26580.
Zadeh J N, Steenberg C D, Bois J S, Wolfe B R, Pierce M B, Khan A R, Dirks R M, Pierce N A. NUPACK: analysis and design of nucleic acid systems. Journal of computational chemistry. 2011 Jan. 15; 32(1):170-3.

8. Sequence Listing

SEQ ID NO: 1-Wild-type Ignavibacterium Cas9 protein
MKKVLGLDLGVSSIGWALIDEDDRKIMGMGSRIIPLTTDDKDEFTKGNTISKNQQRTIKRTQRKGYDRYQLRRQNL
VFVLKQNNMMPDIELVNLPKLELWKLRSDAVNKKISLKELGRILLHLNQKRGYKSSRSESNLDKKDTEYVATVKNRY
ESLKEIGLTIGQKFFEELSKNNFYRIKEQVYPREAYVEEYNKIMKHQQKHYPENISEELINKIRDEIIYYQRKLKSQKGLV
SVCEFEGFWIKLNSNGKEKDLFVGPKVTPKSSPLFQVSRIWETINNISIKRKTGESIEITLDKKKEIFAYMDKNEKLSYP
ELLKILGLKKDDVYGNKNLTNGLLGNKIKTEMMKCISDIDKYSDLFRLELEIKEFDEEVYLYDRTTGEIINSKKKKNIIAAI
EDQPFYKLWHVVYSIPDKETCQKILMSKFGIQEEDAAKLATLDFTKLGFSNKSHRAIRKMLPYLMEGDNDYMARCY
AGYHHTTTITKQENFQRKLLDKLKNLEKNSLRQPIVEKILNQMINVVNAIIDKYGKPDEIRIELARELKQSREERNEAY
RNMNERERENKIIEKELSEFGLRATRNNIIKWRLYHEISNEEKKQNAICIYCGKPISFTAAILGEEVEVEHIIPRSRLFDD
SQSNKTLAHRKCNADKKDQTAYDFMRSKSDTEFNDYVERINTLYKNHVIGKTKRDKLLMSEEKIPMDFIDRQLRQT
QYISKKALELLQNICYNVWATSGNVTAELRHIWGWDEVLENLQLPKYRESGLIEIIEVGDKDNKQKKEKIIGWTKRD
DHRHHAIDALTIACTKQGFIQRFNRLNSGKVRNDMLQEIENAKQNYDKRKNLLENYILSYRPFTTKEVEREAEKILVS
FKAGKKVASTGKRKIKKDGKKIIAQTGIIIPRGPLSEESVYGKIKVIEKEKPLKYLFENPHLIFKPNIKALVEERLYKNNND
PKSAIASLKKEPIYLDKEKTIKLEYGTCYKEEVVIKKPLQALNEKQVEDIVDPIIKQKIKDRLVKFGGKAKEAFKDLENEPI
WYDEEKRIPIKNVRWFTGLSAIEPISKDETGKEIGFVKPGNNHHLAIYIDEEGKKQLSICSFWHAVERKKYGLPVIIKN
PSEVVDFILAEENEDKYPESFLEKLPAGKWTFKESFQQNEMFVLGISKEAFEEAISRNDYSFLSNYLYRVQKIAMIGK
QPNIVFRHHLETQLKDDAYAKKSNRFYLIQSIGALESLYPIKILINCLGEIITNNK*
SEQ ID NO: 2-Nucleic acid sequence encoding wild-type Ignavibacterium Cas9
protein (SEQ ID NO: 1)
ATGAAAAAAGTATTAGGATTAGATCTTGGAGTATCTTCAATAGGCTGGGCTTTAATTGACGAAGATGATAGAA
AAATAATGGGCATGGGTAGTAGAATAATACCATTAACAACTGATGATAAAGACGAGTTTACAAAAGGCAATA
CGATTTCTAAGAATCAGCAACGAACAATTAAAAGAACTCAAAGAAAAGGATACGATCGTTATCAATTAAGAAG
GCAGAATTTAGTTTTCGTGTTGAAACAAAATAATATGATGCCTGATATTGAATTAGTAAATCTTCCAAAACTTG
AATTATGGAAACTAAGAAGTGATGCGGTTAATAAAAAAATATCTTTGAAAGAATTAGGCAGAATCCTACTTCA
CTTAAATCAAAAAAGAGGTTATAAAAGTAGCAGAAGTGAATCAAATTTGGATAAGAAAGATACCGAATATGT
AGCAACAGTAAAAAACAGATATGAAAGCCTAAAAGAAATTGGTTTAACAATAGGACAGAAATTTTTTGAGGA
ATTATCCAAAAACAATTTTTACAGAATAAAAGAACAGGTTTACCCAAGAGAAGCATATGTTGAAGAGTATAAT
AAAATAATGAAGCATCAACAAAAACATTATCCAGAAAATATTTCGGAAGAATTAATTAATAAAATAAGAGACG
AAATAATTTACTATCAACGAAAACTAAAATCGCAAAAGGGATTGGTGTCTGTTTGCGAGTTTGAAGGATTTTG
GATAAAGCTAAATTCAAATGGAAAAGAAAAAGATTTATTTGTTGGTCCAAAAGTAACTCCTAAAAGTTCACCA
TTATTCCAGGTAAGTAGAATTTGGGAAACTATCAATAACATATCAATTAAAAGAAAGACTGGTGAATCCATTG
AAATTACACTGGATAAAAAGAAAGAAATTTTTGCTTATATGGATAAAAATGAAAAATTAAGCTATCCAGAATT
ATTAAAAATTTTAGGGCTTAAAAAAGATGACGTATATGGAAACAAGAATTTAACAAATGGGTTGCTGGGCAAC
AAAATAAAAACAGAAATGATGAAGTGTATTTCAGATATTGATAAGTATTCTGATTTATTCCGATTAGAACTTGA
AATAAAAGAATTCGATGAAGAGGTTTATTTATATGATAGAACAACCGGAGAAATAATAAATTCAAAGAAAAA
AAAGAATATAATAGCAGCAATAGAAGACCAACCATTTTACAAGCTTTGGCATGTTGTTTATTCAATACCCGATA
AAGAAACTTGTCAAAAAATACTTATGTCAAAATTTGGCATACAGGAAGAAGACGCTGCTAAATTAGCAACACT
TGATTTTACTAAACTTGGTTTTTCGAACAAATCCCACCGTGCAATTAGGAAAATGCTTCCTTATCTAATGGAAG
GGGATAACGATTATATGGCCCGTTGTTATGCGGGTTATCATCACACAACAACAATTACAAAACAAGAAAACTT
CCAAAGAAAACTGTTAGATAAATTAAAAAACTTAGAAAAAAATAGCCTGCGCCAGCCGATAGTTGAAAAAATT
CTAAATCAGATGATAAATGTTGTAAATGCAATTATAGACAAATATGGGAAACCGGATGAAATTAGAATTGAAC
TAGCCAGAGAATTAAAACAGAGTAGAGAAGAAAGAAATGAAGCATATAGAAACATGAATGAACGAGAACGT
GAAAATAAAATAATTGAAAAAGAGCTTTCTGAATTTGGACTTCGTGCAACACGAAACAATATTATCAAATGGA
GATTATATCACGAAATTAGCAACGAAGAAAAGAAACAAAATGCAATTTGCATTTATTGTGGCAAACCAATTTC
CTTTACTGCTGCAATATTAGGTGAAGAAGTTGAAGTTGAACACATAATACCAAGGTCAAGGTTATTTGACGAT
TCTCAAAGCAATAAAACACTGGCACATAGAAAATGCAATGCAGATAAGAAAGACCAAACAGCTTATGACTTTA
TGCGTTCAAAATCTGATACTGAATTTAATGATTACGTTGAGCGAATTAATACCCTTTATAAAAATCATGTAATT
GGAAAAACGAAAAGAGATAAACTTTTAATGTCTGAAGAAAAAATTCCTATGGATTTTATTGACAGACAATTAA
GACAAACACAATACATCTCTAAAAAAGCATTAGAGCTTCTTCAGAATATCTGTTATAATGTGTGGGCAACAAG
CGGAAATGTGACCGCCGAGTTGCGCCATATATGGGGATGGGATGAAGTGCTTGAAAATCTTCAATTACCTAA
GTATAGAGAAAGTGGATTAATAGAAATTATTGAAGTTGGAGATAAAGATAATAAACAAAAAAAGGAAAAGAT
AATTGGATGGACCAAAAGAGACGATCATAGACATCATGCAATTGATGCTCTTACCATCGCATGTACCAAACAA
GGATTTATCCAACGCTTTAATAGATTAAATAGTGGGAAAGTACGAAACGATATGCTTCAGGAAATTGAAAACG
CCAAACAGAATTACGATAAAAGAAAAAATCTTTTGGAGAACTATATTCTTTCTTACAGACCATTTACAACAAAG
GAAGTTGAAAGAGAGGCTGAGAAAATACTTGTATCATTCAAAGCCGGCAAAAAGGTTGCATCTACAGGCAAA
AGAAAAATTAAAAAAGATGGCAAAAAAATAATCGCTCAAACTGGTATTATTATTCCAAGAGGACCATTAAGTG
AAGAAAGTGTCTATGGAAAAATAAAAGTAATTGAGAAGGAAAAACCGTTAAAATATTTATTTGAAAATCCACA
CCTCATATTTAAACCAAATATAAAAGCACTTGTAGAAGAAAGACTTTACAAAAACAATAACGACCCTAAAAGT
GCTATAGCTTCATTAAAAAAAGAACCTATTTATCTTGACAAAGAGAAAACAATAAAATTGGAATACGGAACAT
GTTATAAAGAAGAAGTTGTTATAAAAAAACCACTACAAGCTTTGAACGAGAAGCAAGTAGAGGATATTGTTG
ACCCTATAATAAAACAAAAGATTAAGGATCGACTGGTTAAATTTGGTGGCAAAGCCAAAGAAGCATTTAAGG
ATTTAGAAAACGAACCTATTTGGTATGATGAGGAAAAAAGAATTCCAATAAAGAATGTTCGATGGTTTACAGG
ACTTTCAGCAATTGAACCTATAAGCAAGGATGAGACCGGAAAAGAAATTGGATTTGTCAAACCTGGCAATAAT
CATCATCTTGCAATATACATTGATGAAGAAGGGAAAAAACAACTTAGTATATGTTCATTTTGGCATGCTGTAGA
AAGAAAGAAATATGGGTTGCCTGTTATAATAAAAAATCCGTCAGAGGTTGTTGATTTTATACTTGCGGAGGAA
AATGAAGATAAATATCCAGAAAGTTTTCTAGAAAAATTACCCGCTGGGAAATGGACATTTAAAGAAAGCTTTC
AACAAAACGAGATGTTTGTACTTGGAATAAGCAAAGAAGCATTTGAAGAAGCCATTTCGAGAAATGATTATA
GCTTCTTAAGTAATTACTTATATCGTGTTCAAAAGATTGCAATGATAGGCAAACAACCAAATATTGTTTTTAGA
CATCATCTCGAAACTCAGCTTAAGGATGACGCATACGCTAAAAAAAGTAATCGCTTTTATTTAATACAAAGTAT
CGGGGCATTAGAATCATTATATCCAATAAAAATTTTAATTAATTGTTTGGGAGAAATTATTACTAATAATAAAT
AA
SEQ ID NO: 3-Codon optimized nucleic acid sequence encoding wild-type Ignavibacterium Cas9
protein (SEQ ID NO: 1)
ATGAAGAAGGTCCTGGGCTTAGACCTGGGTGTGAGCTCGATTGGTTGGGCGCTGATTGACGAAGACGACCGC
AAGATTATGGGAATGGGATCCCGTATCATTCCGCTGACCACCGATGATAAGGATGAGTTTACAAAGGGTAAC
ACAATCAGCAAAAATCAGCAGCGCACCATCAAGCGCACGCAACGTAAGGGATATGATCGTTATCAGCTGCGC
CGCCAGAATCTGGTGTTTGTTTTAAAACAAAATAACATGATGCCCGATATTGAGCTGGTTAACCTGCCCAAGCT
GGAACTGTGGAAACTGCGTTCTGATGCTGTAAATAAGAAAATCTCTTTAAAAGAACTGGGCCGTATCCTGTTA
CACCTGAATCAGAAACGTGGTTATAAATCATCTCGCTCTGAGTCAAACCTGGACAAGAAGGATACAGAGTATG
TTGCTACGGTCAAAAATCGTTATGAAAGCTTAAAGGAGATCGGCTTAACGATTGGCCAGAAGTTCTTCGAAGA
GTTATCGAAGAACAATTTTTATCGCATCAAGGAACAGGTCTATCCGCGTGAAGCCTACGTCGAGGAATATAAT
AAAATCATGAAACACCAACAGAAACATTACCCCGAGAATATTTCGGAGGAACTGATTAACAAGATCCGTGACG
AAATCATTTACTACCAACGCAAACTGAAATCTCAGAAAGGACTGGTGTCGGTATGCGAGTTTGAGGGATTTTG
GATCAAACTGAACTCGAATGGTAAGGAAAAAGATTTATTTGTCGGTCCAAAGGTAACACCTAAGTCTTCTCCG
CTGTTCCAGGTCTCTCGTATCTGGGAGACTATCAACAACATCAGTATTAAACGTAAGACGGGTGAGTCCATTG
AAATTACGCTGGACAAGAAAAAAGAAATCTTCGCCTACATGGACAAAAATGAAAAGCTGAGTTACCCTGAGC
TGCTGAAAATTCTGGGTCTGAAGAAGGACGACGTTTATGGCAACAAAAATCTGACCAACGGCTTATTAGGTAA
TAAGATCAAAACCGAAATGATGAAATGTATTTCCGACATCGATAAGTATTCAGACCTGTTTCGCCTGGAGCTG
GAGATTAAGGAGTTCGACGAGGAAGTCTACTTATACGATCGCACTACCGGTGAAATCATCAACTCGAAGAAG
AAAAAAAATATCATTGCGGCGATTGAAGACCAACCTTTCTATAAACTGTGGCATGTGGTATACTCGATTCCCG
ACAAGGAGACCTGCCAGAAAATTCTGATGTCTAAGTTCGGCATTCAGGAGGAGGACGCAGCTAAACTGGCGA
CGCTGGATTTCACCAAACTGGGGTTTTCCAATAAGTCACATCGCGCGATTCGCAAAATGCTGCCGTACTTAATG
GAGGGCGATAACGACTATATGGCACGTTGTTATGCTGGTTATCATCATACAACAACCATTACGAAACAAGAGA
ATTTTCAACGCAAATTACTGGATAAGTTAAAAAATCTGGAAAAAAATAGCCTGCGTCAGCCAATTGTGGAGAA
AATCCTGAACCAAATGATTAATGTTGTCAATGCCATTATCGATAAGTATGGTAAACCCGATGAAATCCGCATTG
AATTAGCGCGTGAACTGAAGCAGTCTCGCGAGGAACGTAACGAAGCCTACCGTAATATGAACGAACGTGAGC
GTGAAAACAAAATTATCGAGAAGGAACTGAGTGAATTCGGCCTGCGTGCCACGCGTAACAATATTATCAAAT
GGCGCCTGTACCACGAGATTTCTAATGAAGAGAAAAAGCAGAATGCTATTTGTATCTACTGTGGAAAGCCTAT
TTCATTTACAGCTGCGATTCTGGGAGAGGAAGTAGAAGTTGAACACATCATCCCTCGTAGTCGCCTGTTCGAT
GACTCGCAGAGCAATAAGACCCTGGCGCATCGCAAGTGCAATGCTGATAAGAAGGACCAGACCGCATACGAT
TTTATGCGTTCGAAGTCTGATACTGAATTTAACGACTACGTAGAGCGCATCAATACCCTGTACAAAAACCACGT
CATTGGGAAAACTAAGCGCGACAAACTGCTGATGTCCGAGGAGAAAATTCCAATGGACTTCATCGATCGTCAA
CTGCGCCAGACTCAATACATTTCCAAGAAGGCACTGGAGCTGCTGCAGAACATTTGCTACAATGTTTGGGCTA
CTAGCGGCAATGTTACCGCAGAACTGCGTCACATTTGGGGCTGGGATGAGGTTCTGGAAAACCTGCAGCTGC
CTAAGTACCGTGAATCCGGCTTAATTGAAATTATCGAAGTTGGAGACAAGGACAATAAGCAGAAAAAAGAGA
AGATCATTGGCTGGACTAAGCGCGACGATCATCGCCATCATGCTATTGACGCACTGACAATTGCGTGTACCAA
GCAGGGTTTCATCCAGCGTTTTAATCGTCTGAACAGTGGGAAGGTCCGTAATGACATGCTGCAGGAAATCGA
GAATGCGAAACAGAACTACGATAAGCGCAAAAACTTACTGGAAAACTACATTCTGTCTTATCGTCCTTTCACTA
CTAAAGAAGTTGAGCGCGAGGCAGAAAAAATCTTGGTCTCTTTCAAGGCGGGAAAAAAAGTCGCGTCGACTG
GTAAACGCAAGATCAAGAAAGATGGTAAGAAGATTATCGCGCAAACAGGGATCATCATCCCACGCGGTCCAC
TGAGCGAAGAGAGCGTCTACGGAAAAATCAAGGTCATCGAAAAGGAAAAACCACTGAAATATCTGTTTGAAA
ATCCACATCTGATTTTTAAACCCAATATCAAGGCACTGGTTGAAGAGCGTCTGTACAAAAACAACAATGACCC
GAAAAGTGCTATCGCGTCATTAAAGAAGGAGCCAATTTATTTAGACAAGGAGAAGACCATTAAACTGGAGTA
TGGGACGTGCTACAAGGAAGAGGTCGTCATCAAGAAGCCGTTACAAGCCCTGAATGAGAAACAAGTAGAGG
ACATCGTCGATCCGATCATTAAGCAAAAGATCAAGGACCGCCTGGTGAAGTTCGGCGGTAAGGCAAAAGAAG
CATTTAAGGATCTGGAAAACGAGCCGATCTGGTACGATGAGGAGAAGCGCATCCCGATCAAGAACGTACGCT
GGTTCACTGGTCTGTCGGCTATCGAGCCGATCAGCAAAGATGAAACCGGTAAGGAGATTGGGTTTGTCAAAC
CTGGTAACAATCACCATCTGGCGATTTACATTGACGAGGAGGGGAAGAAGCAGCTGAGCATCTGTAGTTTTTG
GCATGCCGTCGAGCGTAAAAAATACGGACTGCCTGTAATCATTAAAAACCCATCTGAAGTGGTTGATTTCATT
CTGGCCGAGGAAAATGAAGACAAGTATCCAGAGTCCTTTTTAGAGAAGCTGCCCGCGGGGAAGTGGACATTC
AAAGAGTCGTTCCAGCAAAACGAGATGTTCGTCCTGGGTATCTCAAAAGAAGCATTCGAAGAGGCAATTTCGC
GCAATGATTATAGCTTCTTATCGAATTACCTGTACCGTGTGCAAAAAATTGCTATGATCGGGAAGCAGCCCAAT
ATCGTTTTTCGCCATCATCTGGAGACCCAACTGAAGGACGACGCGTATGCCAAAAAGTCGAATCGTTTTTACCT
GATCCAGAGTATTGGTGCCTTAGAATCTTTATATCCTATTAAAATTCTGATTAATTGCCTGGGAGAGATTATCA
CTAATAACAAGTAA
SEQ ID NO: 4-PAM sequence
CCACATCGAA
SEQ ID NO: 5-PAM sequence
AGACATGAAA
SEQ ID NO: 6-PAM motif
NVRNAT, wherein N is any nucleotide, V is A, G or C, and R is G or A.
SEQ ID NO: 7-scaffold sequence portion of sgRNA
GUUGUGAUUUGCUUUCAAAGAAAUUUGAAGCAAAUCACAAUAAGGAUUUUUCCGUUGUGAAAACAUU
UACAGUAGUCCCGAUGCAAACCAUCGGGAUUGUUGUUUU
SEQ ID NO: 8-100-bp DNA target template
CATGGTCAGACAAGCTTACTAGTAAAGGATCCACGGGTACCGAGCTTCCATCC[GGGAATAGTTACATTACTAT
CTGTA]GGACATGAAAGAATTCGTAAT, where the target DNA region is in brackets and the PAM
sequence (which falls within the PAM motif NVRNAT (SEQ ID NO: 6), where N is any nucleotide,
V is A, G or C, and R is G or A) is in bold.
SEQ ID NO: 9-sgRNA sequence
[GGGAAUAGUUACAUUACUAUCUGUA]GUUGUGAUUUGCUUUCAAAGAAAUUUGAAGCAAAUCACAA
UAAGGAUUUUUCCGUUGUGAAAACAUUUACAGUAGUCCCGAUGCAAACCAUCGGGAUUGUUGUUUU,
where the guide sequence is in brackets and the scaffold sequence (SEQ ID NO: 7) is in bold.
SEQ ID NO: 10-PAM sequence
GGACAT
SEQ ID NO: 11-target DNA sequence
GGGAATAGTTACATTACTATCTGTA
SEQ ID NO: 12-starting sequence of PAM
AGACAT
SEQ ID NO: 13-PAM motif
NRRNAT, wherein N is any nucleotide, and R is G or A.
SEQ ID NO: 14-Streptococcus pyogenes
MDKKYSIGLDIGTNSVGWAVITDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKGRDFATVRKVLSMPQ
VNIVKKTEVQTGGFSKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLFTLTNLGAPAAFKYFDT
TIDRKRYTSTKEVLDATLIHQSITGLYETRIDLSQLGGD
SEQ ID NO: 15-Streptococcus thermophilus
MLFNKCIIISINLDFSNKEKCMTKPYSIGLDIGTNSVGWAVITDNYKVPSKKMKVLGNTSKKYIKKNLLGVLLFDSGIT
AEGRRLKRTARRRYTRRRNRILYLQEIFSTEMATLDDAFFQRLDDSFLVPDDKRDSKYPIFGNLVEEKVYHDEFPTIYH
LRKYLADSTKKADLRLVYLALAHMIKYRGHFLIEGEFNSKNNDIQKNFQDFLDTYNAIFESDLSLENSKQLEEIVKDKIS
KLEKKDRILKLFPGEKNSGIFSEFLKLIVGNQADFRKCFNLDEKASLHFSKESYDEDLETLLGYIGDDYSDVFLKAKKLY
DAILLSGFLTVTDNETEAPLSSAMIKRYNEHKEDLALLKEYIRNISLKTYNEVFKDDTKNGYAGYIDGKTNQEDFYVYL
KNLLAEFEGADYFLEKIDREDFLRKQRTFDNGSIPYQIHLQEMRAILDKQAKFYPFLAKNKERIEKILTFRIPYYVGPLA
RGNSDFAWSIRKRNEKITPWNFEDVIDKESSAEAFINRMTSFDLYLPEEKVLPKHSLLYETFNVYNELTKVRFIAESM
RDYQFLDSKQKKDIVRLYFKDKRKVTDKDIIEYLHAIYGYDGIELKGIEKQFNSSLSTYHDLLNIINDKEFLDDSSNEAIIE
EIIHTLTIFEDREMIKQRLSKFENIFDKSVLKKLSRRHYTGWGKLSAKLINGIRDEKSGNTILDYLIDDGISNRNFMQLIH
DDALSFKKKIQKAQIIGDEDKGNIKEVVKSLPGSPAIKKGILQSIKIVDELVKVMGGRKPESIVVEMARENQYTNQGK
SNSQQRLKRLEKSLKELGSKILKENIPAKLSKIDNNALQNDRLYLYYLQNGKDMYTGDDLDIDRLSNYDIDHIIPQAFL
KDNSIDNKVLVSSASNRGKSDDFPSLEVVKKRKTFWYQLLKSKLISQRKFDNLTKAERGGLLPEDKAGFIQRQLVETR
QITKHVARLLDEKFNNKKDENNRAVRTVKIITLKSTLVSQFRKDFELYKVREINDFHHAHDAYLNAVIASALLKKYPKL
EPEFVYGDYPKYNSFRERKSATEKVYFYSNIMNIFKKSISLADGRVIERPLIEVNEETGESVWNKESDLATVRRVLSYP
QVNVVKKVEEQNHGLDRGKPKGLFNANLSSKPKPNSNENLVGAKEYLDPKKYGGYAGISNSFAVLVKGTIEKGAKK
KITNVLEFQGISILDRINYRKDKLNFLLEKGYKDIELIIELPKYSLFELSDGSRRMLASILSTNNKRGEIHKGNQIFLSQKF
VKLLYHAKRISNTINENHRKYVENHKKEFEELFYYILEFNENYVGAKKNGKLLNSAFQSWQNHSIDELCSSFIGPTGSE
RKGLFELTSRGSAADFEFLGVKIPRYRDYTPSSLLKDATLIHQSVTGLYETRIDLAKLGEG
SEQ ID NO: 16-Wolinella succinogenes
MIERILGVDLGISSLGWAIVEYDKDDEAANRIIDCGVRLFTAAETPKKKESPNKARREARGIRRVLNRRRVRMNMIK
KLFLRAGLIQDVDLDGEGGMFYSKANRADVWELRHDGLYRLLKGDELARVLIHIAKHRGYKFIGDDEADEESGKVK
KAGVVLRQNFEAAGCRTVGEWLWRERGANGKKRNKHGDYEISIHRDLLVEEVEAIFVAQQEMRSTIATDALKAAY
REIAFFVRPMQRIEKMVGHCTYFPEERRAPKSAPTAEKFIAISKFFSTVIIDNEGWEQKIIERKTLEELLDFAVSREKVE
FRHLRKFLDLSDNEIFKGLHYKGKPKTAKKREATLFDPNEPTELEFDKVEAEKKAWISLRGAAKLREALGNEFYGRFV
ALGKHADEATKILTYYKDEGQKRRELTKLPLEAEMVERLVKIGFSDFLKLSLKAIRDILPAMESGARYDEAVLMLGVP
HKEKSAILPPLNKTDIDILNPTVIRAFAQFRKVANALVRKYGAFDRVHFELAREINTKGEIEDIKESQRKNEKERKEAA
DWIAETSFQVPLTRKNILKKRLYIQQDGRCAYTGDVIELERLFDEGYCEIDHILPRSRSADDSFANKVLCLARANQQK
TDRTPYEWFGHDAARWNAFETRTSAPSNRVRTGKGKIDRLLKKNFDENSEMAFKDRNLNDTRYMARAIKTYCEQ
YWVFKNSHTKAPVQVRSGKLTSVLRYQWGLESKDRESHTHHAVDAIIIAFSTQGMVQKLSEYYRFKETHREKERPK
LAVPLANFRDAVEEATRIENTETVKEGVEVKRLLISRPPRARVTGQAHEQTAKPYPRIKQVKNKKKWRLAPIDEEKFE
SFKADRVASANQKNFYETSTIPRVDVYHKKGKFHLVPIYLHEMVLNELPNLSLGTNPEAMDENFFKFSIFKDDLISIQ
TQGTPKKPAKIIMGYFKNMHGANMVLSSINNSPCEGFTCTPVSMDKKHKDKCKLCPEENRIAGRCLQGFLDYWS
QEGLRPPRKEFECDQGVKFALDVKKYQIDPLGYYYEVKQEKRLGTIPQMRSAKKLVKK
SEQ ID NO: 17-Neisseria meningitidis
MAAFKPNPINYILGLDIGIASVGWAMVEIDEDENPICLIDLGVRVFERAEVPKTGDSLAMARRLARSVRRLTRRRAH
RLLRARRLLKREGVLQAADFDENGLIKSLPNTPWQLRAAALDRKLTPLEWSAVLLHLIKHRGYLSQRKNEGETADKE
LGALLKGVADNAHALQTGDFRTPAELALNKFEKESGHIRNQRGDYSHTFSRKDLQAELILLFEKQKEFGNPHVSGGL
KEGIETLLMTQRPALSGDAVQKMLGHCTFEPAEPKAAKNTYTAERFIWLTKLNNLRILEQGSERPLTDTERATLMDE
PYRKSKLTYAQARKLLGLEDTAFFKGLRYGKDNAEASTLMEMKAYHAISRALEKEGLKDKKSPLNLSPELQDEIGTAF
SLFKTDEDITGRLKDRIQPEILEALLKHISFDKFVQISLKALRRIVPLMEQGKRYDEACAEIYGDHYGKKNTEEKIYLPPI
PADEIRNPVVLRALSQARKVINGVVRRYGSPARIHIETAREVGKSFKDRKEIEKRQEENRKDREKAAAKFREYFPNFV
GEPKSKDILKLRLYEQQHGKCLYSGKEINLGRLNEKGYVEIDHALPFSRTWDDSFNNKVLVLGSENQNKGNQTPYEY
FNGKDNSREWQEFKARVETSRFPRSKKQRILLQKFDEDGFKERNLNDTRYVNRFLCQFVADRMRLTGKGKKRVFA
SNGQITNLLRGFWGLRKVRAENDRHHALDAVVVACSTVAMQQKITRFVRYKEMNAFDGKTIDKETGEVLHQKTH
FPQPWEFFAQEVMIRVFGKPDGKPEFEEADTPEKLRTLLAEKLSSRPEAVHEYVTPLFVSRAPNRKMSGQGHMET
VKSAKRLDEGVSVLRVPLTQLKLKDLEKMVNREREPKLYEALKARLEAHKDDPAKAFAEPFYKYDKAGNRTQQVKA
VRVEQVQKTGVWVRNHNGIADNATMVRVDVFEKGDKYYLVPIYSWQVAKGILPDRAVVQGKDEEDWQLIDDSF
NFKFSLHPNDLVEVITKKARMFGYFASCHRGTGNINIRIHDLDHKIGKNGILEGIGVKTALSFQKYQIDELGKEIRPCR
LKKRPPVR
SEQ ID NO: 18-Actinomyces naeslundii
MWYASLMSAHHLRVGIDVGTHSVGLATLRVDDHGTPIELLSALSHIHDSGVGKEGKKDHDTRKKLSGIARRARRLL
HHRRTQLQQLDEVLRDLGFPIPTPGEFLDLNEQTDPYRVWRVRARLVEEKLPEELRGPAISMAVRHIARHRGWRN
PYSKVESLLSPAEESPFMKALRERILATTGEVLDDGITPGQAMAQVALTHNISMRGPEGILGKLHQSDNANEIRKICA
RQGVSPDVCKQLLRAVFKADSPRGSAVSRVAPDPLPGQGSFRRAPKCDPEFQRFRIISIVANLRISETKGENRPLTAD
ERRHVVTFLTEDSQADLTWVDVAEKLGVHRRDLRGTAVHTDDGERSAARPPIDATDRIMRQTKISSLKTWWEEA
DSEQRGAMIRYLYEDPTDSECAEIIAELPEEDQAKLDSLHLPAGRAAYSESLTALSDHMLATTDDLHEARKRLFGVD
DSWAPPAEAINAPVGNPSVDRTLKIVGRYLSAVESMWGTPEVIHVEHVRDGFTSERMADERDKANRRRYNDNQ
EAMKKIQRDYGKEGYISRGDIVRLDALELQGCACLYCGTTIGYHTCQLDHIVPQAGPGSNNRRGNLVAVCERCNRS
KSNTPFAVWAQKCGIPHVGVKEAIGRVRGWRKQTPNTSSEDLTRLKKEVIARLRRTQEDPEIDERSMESVAWMA
NELHHRIAAAYPETTVMVYRGSITAAARKAAGIDSRINLIGEKGRKDRIDRRHHAVDASVVALMEASVAKTLAERSS
LRGEQRLTGKEQTWKQYTGSTVGAREHFEMWRGHMLHLTELFNERLAEDKVYVTQNIRLRLSDGNAHTVNPSKL
VSHRLGDGLTVQQIDRACTPALWCALTREKDFDEKNGLPAREDRAIRVHGHEIKSSDYIQVFSKRKKTDSDRDETPF
GAIAVRGGFVEIGPSIHHARIYRVEGKKPVYAMLRVFTHDLLSQRHGDLFSAVIPPQSISMRCAEPKLRKAITTGNAT
YLGWVVVGDELEINVDSFTKYAIGRFLEDFPNTTRWRICGYDTNSKLTLKPIVLAAEGLENPSSAVNEIVELKGWRV
AINVLTKVHPTVVRRDALGRPRYSSRSNLPTSWTIE
SEQ ID NO: 19-Geobacillus stearothermophilus
MRYKIGLDIGITSVGWAVMNLDIPRIEDLGVRIFDRAENPQTGESLALPRRLARSARRRLRRRKHRLERIRRLVIREGI
LTKEELDKLFEEKHEIDVWQLRVEALDRKLNNDELARVLLHLAKRRGFKSNRKSERSNKENSTMLKHIEENRAILSSY
RTVGEMIVKDPKFALHKRNKGENYTNTIARDDLEREIRLIFSKQREFGNMSCTEEFENEYITIWASQRPVASKDDIEK
KVGFCTFEPKEKRAPKATYTFQSFIAWEHINKLRLISPSGARGLTDEERRLLYEQAFQKNKITYHDIRTLLHLPDDTYFK
GIVYDRGESRKQNENIRFLELDAYHQIRKAVDKVYGKGKSSSFLPIDFDTFGYALTLFKDDADIHSYLRNEYEQNGKR
MPNLANKVYDNELIEELLNLSFTKFGHLSLKALRSILPYMEQGEVYSSACERAGYTFTGPKKKQKTMLLPNIPPIANP
VVMRALTQARKVVNAIIKKYGSPVSIHIELARDLSQTFDERRKTKKEQDENRKKNETAIRQLMEYGLTLNPTGHDIV
KFKLWSEQNGRCAYSLQPIEIERLLEPGYVEVDHVIPYSRSLDDSYTNKVLVLTRENREKGNRIPAEYLGVGTERWQ
QFETFVLTNKQFSKKKRDRLLRLHYDENEETEFKNRNLNDTRYISRFFANFIREHLKFAESDDKQKVYTVNGRVTAHL
RSRWEFNKNREESDLHHAVDAAIVACTTPSDIAKVTAFYQRREQNKELAKKTEPHFPQPWPHFADELRARLSKHPK
ESIKALNLGNYDDQKLESLQPVFVSRMPKRSVTGAAHQETLRRYVGIDERSGKIQTVVKTKLSEIKLDASGHFPMYG
KESDPRTYEAIRQRLLEHNNDPKKAFQEPLYKPKKNGEPGPVIRTVKIIDTKNQVIPLNDGKTVAYNSNIVRVDVFEK
DGKYYCVPVYTMDIMKGILPNKAIEPNKPYSEWKEMTEDYTFRFSLYPNDLIRIELPREKTVKTAAGEEINVKDVFVY
YKTIDSANGGLELISHDHRFSLRGVGSRTLKRFEKYQVDVLGNIYKVRGEKRVGLASSAHSKTGETVRPLQSTRD

What is claimed is:

1. An isolated clustered regularly interspaced short palindromic repeats (CRISPR)-associated (Cas) 9 protein variant comprising the sequence of SEQ ID NO: 1 or an enzymatically active variant or fragment thereof, wherein the enzymatically active variant or fragment has Cas9 nuclease activity at 70° C. or above.

2. An isolated Cas9 protein variant comprising a sequence having at least 75% sequence identity to the sequence of a wild-type Cas9 protein, wherein the Cas protein variant has at least one amino acid substitution relative to the sequence of the wild-type Cas9 protein, and wherein the wild-type Cas9 protein has a sequence of SEQ ID NO:1.

3. The isolated Cas9 protein variant of claim 2, wherein the isolated Cas9 protein variant is a fragment of the wild-type Cas9 protein.

4. The isolated Cas9 protein variant of any one of claims 1 to 3, wherein the isolated Cas9 protein variant has nuclease activity at a temperature of between 20° C. and 100° C.

5. The isolated Cas9 protein variant of any one of claims 1 to 3, wherein the isolated Cas9 protein variant has nuclease activity at a temperature of at least 70° C.

6. The isolated Cas9 protein variant of any one of claims 1 to 5, wherein the isolated Cas9 protein variant forms a ribonucleoprotein complex with a single-guide RNA (sgRNA), wherein the sgRNA comprises a guide sequence and a scaffold sequence.

7. The isolated Cas9 protein variant of claim 6, wherein the scaffold sequence has at least 75% sequence identity to the sequence of GUUGUGAUUUGCUUUCAAAGAAAUUUGAAGCAAAUCACAAUAAGGAUUUUUCCGUUGUGAAAACAUU UACAGUAGUCCCGAUGCAAACCAUCGGGAUUGUUGUUUU (SEQ ID NO:7).

8. The isolated Cas9 protein variant of claim 6 or 7, wherein the guide sequence has at least 22 nucleotides.

9. The isolated Cas9 protein variant of any one of claims 6 to 8, wherein the guide sequence has between 22 and 25 nucleotides.

10. The isolated Cas9 protein variant of any one of claims 1 to 9, wherein the isolated Cas9 protein variant recognizes an adenine-rich protospacer adjacent motif (PAM) sequence.

11. The isolated Cas9 protein variant of claim 10, wherein the adenine-rich PAM sequence comprises at least 40% adenine in its sequence.

12. The isolated Cas9 protein variant of claim 10 or 11, wherein the adenine-rich PAM sequence has at least 70% sequence identity to the sequence of CCACATCGAA (SEQ ID NO:4) or AGACATGAAA (SEQ ID NO:5).

13. The isolated Cas9 protein variant of any one of claims 1 to 9, wherein the isolated Cas9 protein variant binds a PAM motif having the sequence of NVRNAT (SEQ ID NO:6), wherein N is any nucleotide, V is A, G or C, and R is G or A.

14. The isolated Cas9 protein variant of any one of claims 1 to 9, wherein the isolated Cas9 protein variant binds a PAM motif having the sequence of NRRNAT (SEQ ID NO:13), wherein N is any nucleotide and R is G or A.

15. The isolated Cas9 protein variant of claim 13 or 14, wherein the PAM motif has the sequence of GGACAT (SEQ ID NO:10).

16. A ribonucleoprotein complex comprising:
(1) an isolated Cas9 protein variant comprising a sequence having at least 75% sequence identity to the sequence of a wild-type Cas9 protein, and
(2) an sgRNA comprising a guide sequence and a scaffold sequence,
wherein the scaffold sequence has at least 75% sequence identity to the sequence of SEQ ID NO:7.

17. The ribonucleoprotein complex of claim 16, wherein the guide sequence has at least 22 nucleotides.

18. The ribonucleoprotein complex of claim 17, wherein the guide sequence has between 22 and 25 nucleotides.

19. A composition comprising:
(1) a ribonucleoprotein complex comprising:
(a) an isolated Cas9 protein variant comprising a sequence having at least 75% sequence identity to the sequence of a wild-type Cas9 protein, and
(b) an sgRNA comprising a guide sequence and a scaffold sequence, and
(2) a ribosomal complementary DNA (cDNA),
wherein the scaffold sequence has at least 75% sequence identity to the sequence of SEQ ID NO:7.

20. The ribonucleoprotein complex of claim 19, wherein the ribosomal cDNA is generated in a polymerase chain reaction (PCR).

21. The ribonucleoprotein complex of any one of claims 16 to 20, wherein the isolated Cas9 protein variant comprises at least one amino acid substitution relative to the sequence of the wild-type Cas9 protein.

22. The ribonucleoprotein complex of any one of claims 16 to 21, wherein the isolated Cas9 protein variant comprises a fragment of the wild-type Cas9 protein.

23. The ribonucleoprotein complex of any one of claims 16 to 22, wherein the wild-type Cas9 protein has the sequence of SEQ ID NO:1.

24. The ribonucleoprotein complex of any one of claims 16 to 23, wherein the isolated Cas9 protein variant has nuclease activity at a temperature of between 20° C. and 100° C.

25. The ribonucleoprotein complex of any one of claims 16 to 23, wherein the isolated Cas9 protein variant has nuclease activity at a temperature of at least 70° C.

26. The ribonucleoprotein complex of any one of claims 16 to 25, wherein the isolated Cas9 protein variant recognizes an adenine-rich protospacer adjacent motif (PAM) sequence.

27. The ribonucleoprotein complex of claim 26, wherein the adenine-rich PAM sequence comprises at least 40% adenine in its sequence.

28. The ribonucleoprotein complex of claim 26 or 27, wherein the adenine-rich PAM sequence has at least 70% sequence identity to the sequence of CCACATCGAA (SEQ ID NO:4) or AGACATGAAA (SEQ ID NO:5).

29. The ribonucleoprotein complex of any one of claims 16 to 25, wherein the isolated Cas9 protein variant recognizes a PAM sequence having the sequence of NVRNAT (SEQ ID NO:6), wherein N is any nucleotide, V is A, G or C, and R is G or A.

30. The ribonucleoprotein complex of any one of claims 16 to 25, wherein the isolated Cas9 protein variant recognizes a PAM sequence having the sequence of NRRNAT (SEQ ID NO:13), wherein N is any nucleotide and R is G or A.

31. A cell comprising the ribonucleoprotein complex of any one of claims 16 to 30.

32. A method of altering the genome of a cell, comprising contacting the cell with:
(1) an isolated Cas9 protein variant comprising a sequence having at least 75% sequence identity to the sequence of a wild-type Cas9 protein, and
(2) an sgRNA comprising a guide sequence and a scaffold sequence,
wherein the scaffold sequence has at least 75% sequence identity to the sequence of SEQ ID NO:7,
wherein the isolated Cas9 protein variant interacts with the sgRNA and a target DNA within the cell, and
wherein the guide sequence in the sgRNA comprises a region complementary to a region of the target DNA.

33. The method of claim 32, wherein the isolated Cas9 protein variant recognizes an adenine-rich PAM sequence.

34. The method of claim 33, wherein the adenine-rich PAM sequence comprises at least 40% adenine in its sequence.

35. The method of claim 33 or 34, wherein the adenine-rich PAM sequence has at least 70% sequence identity to the sequence of CCACATCGAA (SEQ ID NO:4) or AGACATGAAA (SEQ ID NO:5).

36. The method of claim 32, wherein the isolated Cas9 protein variant recognizes a PAM sequence having the sequence of NVRNAT (SEQ ID NO:6), wherein N is any nucleotide, V is A, G or C, and R is G or A.

37. The method of claim 32, wherein the isolated Cas9 protein variant recognizes a PAM sequence having the sequence of NRRNAT (SEQ ID NO:13), wherein N is any nucleotide and R is G or A.


SEQUENCE LISTING (20 sequences)

SEQ ID NO: 1 - protein, 1240 aa, Ignavibacterium sp.
MKKVLGLDLGVSSIGWALIDEDDRKIMGMGSRIIPLTTDDKDEFTKGNTISKNQQRTIKRTQRKGYDRYQLRRQNLVFVLKQNNMMPDIELVNLPKLELWKLRSDAVNKKISLKELGRILLHLNQKRGYKSSRSESNLDKKDTEYVATVKNRYESLKEIGLTIGQKFFEELSKNNFYRIKEQVYPREAYVEEYNKIMKHQQKHYPENISEELINKIRDEIIYYQRKLKSQKGLVSVCEFEGFWIKLNSNGKEKDLFVGPKVTPKSSPLFQVSRIWETINNISIKRKTGESIEITLDKKKEIFAYMDKNEKLSYPELLKILGLKKDDVYGNKNLTNGLLGNKIKTEMMKCISDIDKYSDLFRLELEIKEFDEEVYLYDRTTGEIINSKKKKNIIAAIEDQPFYKLWHVVYSIPDKETCQKILMSKFGIQEEDAAKLATLDFTKLGFSNKSHRAIRKMLPYLMEGDNDYMARCYAGYHHTTTITKQENFQRKLLDKLKNLEKNSLRQPIVEKILNQMINVVNAIIDKYGKPDEIRIELARELKQSREERNEAYRNMNERERENKIIEKELSEFGLRATRNNIIKWRLYHEISNEEKKQNAICIYCGKPISFTAAILGEEVEVEHIIPRSRLFDDSQSNKTLAHRKCNADKKDQTAYDFMRSKSDTEFNDYVERINTLYKNHVIGKTKRDKLLMSEEKIPMDFIDRQLRQTQYISKKALELLQNICYNVWATSGNVTAELRHIWGWDEVLENLQLPKYRESGLIEIIEVGDKDNKQKKEKIIGWTKRDDHRHHAIDALTIACTKQGFIQRFNRLNSGKVRNDMLQEIENAKQNYDKRKNLLENYILSYRPFTTKEVEREAEKILVSFKAGKKVASTGKRKIKKDGKKIIAQTGIIIPRGPLSEESVYGKIKVIEKEKPLKYLFENPHLIFKPNIKALVEERLYKNNNDPKSAIASLKKEPIYLDKEKTIKLEYGTCYKEEVVIKKPLQALNEKQVEDIVDPIIKQKIKDRLVKFGGKAKEAFKDLENEPIWYDEEKRIPIKNVRWFTGLSAIEPISKDETGKEIGFVKPGNNHHLAIYIDEEGKKQLSICSFWHAVERKKYGLPVIIKNPSEVVDFILAEENEDKYPESFLEKLPAGKWTFKESFQQNEMFVLGISKEAFEEAISRNDYSFLSNYLYRVQKIAMIGKQPNIVFRHHLETQLKDDAYAKKSNRFYLIQSIGALESLYPIKILINCLGEIITNNK

SEQ ID NO: 2 - DNA, 3723 bp, Ignavibacterium sp.
atgaaaaaagtattaggattagatcttggagtatcttcaataggctgggctttaattgacgaagatgatagaaaaataatgggcatgggtagtagaataataccattaacaactgatgataaagacgagtttacaaaaggcaatacgatttctaagaatcagcaacgaacaattaaaagaactcaaagaaaaggatacgatcgttatcaattaagaaggcagaatttagttttcgtgttgaaacaaaataatatgatgcctgatattgaattagtaaatcttccaaaacttgaattatggaaactaagaagtgatgcggttaataaaaaaatatctttgaaagaattaggcagaatcctacttcacttaaatcaaaaaagaggttataaaagtagcagaagtgaatcaaatttggataagaaagataccgaatatgtagcaacagtaaaaaacagatatgaaagcctaaaagaaattggtttaacaataggacagaaattttttgaggaattatccaaaaacaatttttacagaataaaagaacaggtttacccaagagaagcatatgttgaagagtataataaaataatgaagcatcaacaaaaacattatccagaaaatatttcggaagaattaattaataaaataagagacgaaataatttactatcaacgaaaactaaaatcgcaaaagggattggtgtctgtttgcgagtttgaaggattttggataaagctaaattcaaatggaaaagaaaaagatttatttgttggtccaaaagtaactcctaaaagttcaccattattccaggtaagtagaatttgggaaactatcaataacatatcaattaaaagaaagactggtgaatccattgaaattacactggataaaaagaaagaaatttttgcttatatggataaaaatgaaaaattaagctatccagaattattaaaaattttagggcttaaaaaagatgacgtatatggaaacaagaatttaacaaatgggttgctgggcaacaaaataaaaacagaaatgatgaagtgtatttcagatattgataagtattctgatttattccgattagaacttgaaataaaagaattcgatgaagaggtttatttatatgatagaacaaccggagaaataataaattcaaagaaaaaaaagaatataatagcagcaatagaagaccaaccattttacaagctttggcatgttgtttattcaatacccgataaagaaacttgtcaaaaaatacttatgtcaaaatttggcatacaggaagaagacgctgctaaattagcaacacttgattttactaaacttggtttttcgaacaaatcccaccgtgcaattaggaaaatgcttccttatctaatggaaggggataacgattatatggcccgttgttatgcgggttatcatcacacaacaacaattacaaaacaagaaaacttccaaagaaaactgttagataaattaaaaaacttagaaaaaaatagcctgcgccagccgatagttgaaaaaattctaaatcagatgataaatgttgtaaatgcaattatagacaaatatgggaaaccggatgaaattagaattgaactagccagagaattaaaacagagtagagaagaaagaaatgaagcatatagaaacatgaatgaacgagaacgtgaaaataaaataattgaaaaagagctttctgaatttggacttcgtgcaacacgaaacaatattatcaaatggagattatatcacgaaattagcaacgaagaaaagaaacaaaatgcaatttgcatttattgtggcaaaccaatttcctttactgctgcaatattaggtgaagaagttgaagttgaacacataataccaaggtcaaggttatttgacgattctcaaagcaataaaacactggcacatagaaaatgcaatgcagataagaaagaccaaacagcttatgactttatgcgttcaaaatctgatactgaatttaatgattacgttgagcgaattaataccctttataaaaatcatgtaattggaaaaacgaaaagagataaacttttaatgtctgaagaaaaaattcctatggattttattgacagacaattaagacaaacacaatacatctctaaaaaagcattagagcttcttcagaatatctgttataatgtgtgggcaacaagcggaaatgtgaccgccgagttgcgccatatatggggatgggatgaagtgcttgaaaatcttcaattacctaagtatagagaaagtggattaatagaaattattgaagttggagataaagataataaacaaaaaaaggaaaagataattggatggaccaaaagagacgatcatagacatcatgcaattgatgctcttaccatcgcatgtaccaaacaaggatttatccaacgctttaatagattaaatagtgggaaagtacgaaacgatatgcttcaggaaattgaaaacgccaaacagaattacgataaaagaaaaaatcttttggagaactatattctttcttacagaccatttacaacaaaggaagttgaaagagaggctgagaaaatacttgtatcattcaaagccggcaaaaaggttgcatctacaggcaaaagaaaaattaaaaaagatggcaaaaaaataatcgctcaaactggtattattattccaagaggaccattaagtgaagaaagtgtctatggaaaaataaaagtaattgagaaggaaaaaccgttaaaatatttatttgaaaatccacacctcatatttaaaccaaatataaaagcacttgtagaagaaagactttacaaaaacaataacgaccctaaaagtgctatagcttcattaaaaaaagaacctatttatcttgacaaagagaaaacaataaaattggaatacggaacatgttataaagaagaagttgttataaaaaaaccactacaagctttgaacgagaagcaagtagaggatattgttgaccctataataaaacaaaagattaaggatcgactggttaaatttggtggcaaagccaaagaagcatttaaggatttagaaaacgaacctatttggtatgatgaggaaaaaagaattccaataaagaatgttcgatggtttacaggactttcagcaattgaacctataagcaaggatgagaccggaaaagaaattggatttgtcaaacctggcaataatcatcatcttgcaatatacattgatgaagaagggaaaaaacaacttagtatatgttcattttggcatgctgtagaaagaaagaaatatgggttgcctgttataataaaaaatccgtcagaggttgttgattttatacttgcggaggaaaatgaagataaatatccagaaagttttctagaaaaattacccgctgggaaatggacatttaaagaaagctttcaacaaaacgagatgtttgtacttggaataagcaaagaagcatttgaagaagccatttcgagaaatgattatagcttcttaagtaattacttatatcgtgttcaaaagattgcaatgataggcaaacaaccaaatattgtttttagacatcatctcgaaactcagcttaaggatgacgcatacgctaaaaaaagtaatcgcttttatttaatacaaagtatcggggcattagaatcattatatccaataaaaattttaattaattgtttgggagaaattattactaataataaataa

SEQ ID NO: 3 - DNA, 3723 bp, Artificial Sequence
  Description of Artificial Sequence Synthetic polynucleotide
atgaagaaggtcctgggcttagacctgggtgtgagctcgattggttgggcgctgattgacgaagacgaccgcaagattatgggaatgggatcccgtatcattccgctgaccaccgatgataaggatgagtttacaaagggtaacacaatcagcaaaaatcagcagcgcaccatcaagcgcacgcaacgtaagggatatgatcgttatcagctgcgccgccagaatctggtgtttgttttaaaacaaaataacatgatgcccgatattgagctggttaacctgcccaagctggaactgtggaaactgcgttctgatgctgtaaataagaaaatctctttaaaagaactgggccgtatcctgttacacctgaatcagaaacgtggttataaatcatctcgctctgagtcaaacctggacaagaaggatacagagtatgttgctacggtcaaaaatcgttatgaaagcttaaaggagatcggcttaacgattggccagaagttcttcgaagagttatcgaagaacaatttttatcgcatcaaggaacaggtctatccgcgtgaagcctacgtcgaggaatataataaaatcatgaaacaccaacagaaacattaccccgagaatatttcggaggaactgattaacaagatccgtgacgaaatcatttactaccaacgcaaactgaaatctcagaaaggactggtgtcggtatgcgagtttgagggattttggatcaaactgaactcgaatggtaaggaaaaagatttatttgtcggtccaaaggtaacacctaagtcttctccgctgttccaggtctctcgtatctgggagactatcaacaacatcagtattaaacgtaagacgggtgagtccattgaaattacgctggacaagaaaaaagaaatcttcgcctacatggacaaaaatgaaaagctgagttaccctgagctgctgaaaattctgggtctgaagaaggacgacgtttatggcaacaaaaatctgaccaacggcttattaggtaataagatcaaaaccgaaatgatgaaatgtatttccgacatcgataagtattcagacctgtttcgcctggagctggagattaaggagttcgacgaggaagtctacttatacgatcgcactaccggtgaaatcatcaactcgaagaagaaaaaaaatatcattgcggcgattgaagaccaacctttctataaactgtggcatgtggtatactcgattcccgacaaggagacctgccagaaaattctgatgtctaagttcggcattcaggaggaggacgcagctaaactggcgacgctggatttcaccaaactggggttttccaataagtcacatcgcgcgattcgcaaaatgctgccgtacttaatggagggcgataacgactatatggcacgttgttatgctggttatcatcatacaacaaccattacgaaacaagagaattttcaacgcaaattactggataagttaaaaaatctggaaaaaaatagcctgcgtcagccaattgtggagaaaatcctgaaccaaatgattaatgttgtcaatgccattatcgataagtatggtaaacccgatgaaatccgcattgaattagcgcgtgaactgaagcagtctcgcgaggaacgtaacgaagcctaccgtaatatgaacgaacgtgagcgtgaaaacaaaattatcgagaaggaactgagtgaattcggcctgcgtgccacgcgtaacaatattatcaaatggcgcctgtaccacgagatttctaatgaagagaaaaagcagaatgctatttgtatctactgtggaaagcctatttcatttacagctgcgattctgggagaggaagtagaagttgaacacatcatccctcgtagtcgcctgttcgatgactcgcagagcaataagaccctggcgcatcgcaagtgcaatgctgataagaaggaccagaccgcatacgattttatgcgttcgaagtctgatactgaatttaacgactacgtagagcgcatcaataccctgtacaaaaaccacgtcattgggaaaactaagcgcgacaaactgctgatgtccgaggagaaaattccaatggacttcatcgatcgtcaactgcgccagactcaatacatttccaagaaggcactggagctgctgcagaacatttgctacaatgtttgggctactagcggcaatgttaccgcagaactgcgtcacatttggggctgggatgaggttctggaaaacctgcagctgcctaagtaccgtgaatccggcttaattgaaattatcgaagttggagacaaggacaataagcagaaaaaagagaagatcattggctggactaagcgcgacgatcatcgccatcatgctattgacgcactgacaattgcgtgtaccaagcagggtttcatccagcgttttaatcgtctgaacagtgggaaggtccgtaatgacatgctgcaggaaatcgagaatgcgaaacagaactacgataagcgcaaaaacttactggaaaactacattctgtcttatcgtcctttcactactaaagaagttgagcgcgaggcagaaaaaatcttggtctctttcaaggcgggaaaaaaagtcgcgtcgactggtaaacgcaagatcaagaaagatggtaagaagattatcgcgcaaacagggatcatcatcccacgcggtccactgagcgaagagagcgtctacggaaaaatcaaggtcatcgaaaaggaaaaaccactgaaatatctgtttgaaaatccacatctgatttttaaacccaatatcaaggcactggttgaagagcgtctgtacaaaaacaacaatgacccgaaaagtgctatcgcgtcattaaagaaggagccaatttatttagacaaggagaagaccattaaactggagtatgggacgtgctacaaggaagaggtcgtcatcaagaagccgttacaagccctgaatgagaaacaagtagaggacatcgtcgatccgatcattaagcaaaagatcaaggaccgcctggtgaagttcggcggtaaggcaaaagaagcatttaaggatctggaaaacgagccgatctggtacgatgaggagaagcgcatcccgatcaagaacgtacgctggttcactggtctgtcggctatcgagccgatcagcaaagatgaaaccggtaaggagattgggtttgtcaaacctggtaacaatcaccatctggcgatttacattgacgaggaggggaagaagcagctgagcatctgtagtttttggcatgccgtcgagcgtaaaaaatacggactgcctgtaatcattaaaaacccatctgaagtggttgatttcattctggccgaggaaaatgaagacaagtatccagagtcctttttagagaagctgcccgcggggaagtggacattcaaagagtcgttccagcaaaacgagatgttcgtcctgggtatctcaaaagaagcattcgaagaggcaatttcgcgcaatgattatagcttcttatcgaattacctgtaccgtgtgcaaaaaattgctatgatcgggaagcagcccaatatcgtttttcgccatcatctggagacccaactgaaggacgacgcgtatgccaaaaagtcgaatcgtttttacctgatccagagtattggtgccttagaatctttatatcctattaaaattctgattaattgcctgggagagattatcactaataacaagtaa

SEQ ID NO: 4 - DNA, 10 bp, Artificial Sequence
  Description of Artificial Sequence Synthetic oligonucleotide
ccacatcgaa

SEQ ID NO: 5 - DNA, 10 bp, Artificial Sequence
  Description of Artificial Sequence Synthetic oligonucleotide
agacatgaaa

SEQ ID NO: 6 - DNA, 6 bp, Artificial Sequence
  Description of Artificial Sequence Synthetic oligonucleotide
  modified_base (1)..(1): a, c, t, g, unknown or other
  modified_base (4)..(4): a, c, t, g, unknown or other
nvrnat

SEQ ID NO: 7 - RNA, 106 nt, Artificial Sequence
  Description of Artificial Sequence Synthetic polynucleotide
guugugauuugcuuucaaagaaauuugaagcaaaucacaauaaggauuuuuccguugugaaaacauuuacaguagucccgaugcaaaccaucgggauuguuguuuu

SEQ ID NO: 8 - DNA, 99 bp, Artificial Sequence
  Description of Artificial Sequence Synthetic oligonucleotide
catggtcagacaagcttactagtaaaggatccacgggtaccgagcttccatccgggaatagttacattactatctgtaggacatgaaagaattcgtaat

SEQ ID NO: 9 - RNA, 131 nt, Artificial Sequence
  Description of Artificial Sequence Synthetic polynucleotide
gggaauaguuacauuacuaucuguaguugugauuugcuuucaaagaaauuugaagcaaaucacaauaaggauuuuuccguugugaaaacauuuacaguagucccgaugcaaaccaucgggauuguuguuuu

SEQ ID NO: 10 - DNA, 6 bp, Artificial Sequence
  Description of Artificial Sequence Synthetic oligonucleotide
ggacat

SEQ ID NO: 11 - DNA, 25 bp, Artificial Sequence
  Description of Artificial Sequence Synthetic oligonucleotide
gggaatagttacattactatctgta

SEQ ID NO: 12 - DNA, 6 bp, Artificial Sequence
  Description of Artificial Sequence Synthetic oligonucleotide
agacat

SEQ ID NO: 13 - DNA, 6 bp, Artificial Sequence
  Description of Artificial Sequence Synthetic oligonucleotide
  modified_base (1)..(1): a, c, t, g, unknown or other
  modified_base (4)..(4): a, c, t, g, unknown or other
nrrnat

SEQ ID NO: 14 - protein, 1368 aa, Streptococcus pyogenes [sequence omitted]

SEQ ID NO: 15 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 16 - protein, 1059 aa, Wolinella succinogenes
MIERILGVDLGISSLGWAIVEYDKDDEAANRIIDCGVRLFTAAETPKKKESPNKARREARGIRRVLNRRRVRMNMIKKLFLRAGLIQDVDLDGEGGMFYSKANRADVWELRHDGLYRLLKGDELARVLIHIAKHRGYKFIGDDEADEESGKVKKAGVVLRQNFEAAGCRTVGEWLWRERGANGKKRNKHGDYEISIHRDLLVEEVEAIFVAQQEMRSTIATDALKAAYREIAFFVRPMQRIEKMVGHCTYFPEERRAPKSAPTAEKFIAISKFFSTVIIDNEGWEQKIIERKTLEELLDFAVSREKVEFRHLRKFLDLSDNEIFKGLHYKGKPKTAKKREATLFDPNEPTELEFDKVEAEKKAWISLRGAAKLREALGNEFYGRFVALGKHADEATKILTYYKDEGQKRRELTKLPLEAEMVERLVKIGFSDFLKLSLKAIRDILPAMESGARYDEAVLMLGVPHKEKSAILPPLNKTDIDILNPTVIRAFAQFRKVANALVRKYGAFDRVHFELAREINTKGEIEDIKESQRKNEKERKEAADWIAETSFQVPLTRKNILKKRLYIQQDGRCAYTGDVIELERLFDEGYCEIDHILPRSRSADDSFANKVLCLARANQQKTDRTPYEWFGHDAARWNAFETRTSAPSNRVRTGKGKIDRLLKKNFDENSEMAFKDRNLNDTRYMARAIKTYCEQYWVFKNSHTKAPVQVRSGKLTSVLRYQWGLESKDRESHTHHAVDAIIIAFSTQGMVQKLSEYYRFKETHREKERPKLAVPLANFRDAVEEATRIENTETVKEGVEVKRLLISRPPRARVTGQAHEQTAKPYPRIKQVKNKKKWRLAPIDEEKFESFKADRVASANQKNFYETSTIPRVDVYHKKGKFHLVPIYLHEMVLNELPNLSLGTNPEAMDENFFKFSIFKDDLISIQTQGTPKKPAKIIMGYFKNMHGANMVLSSINNSPCEGFTCTPVSMDKKHKDKCKLCPEENRIAGRCLQGFLDYWSQEGLRPPRKEFECDQGVKFALDVKKYQIDPLGYYYEVKQEKRLGTIPQMRSAKKLVKK

SEQ ID NO: 17 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 18 - protein, 1100 aa, Actinomyces naeslundii
MWYASLMSAHHLRVGIDVGTHSVGLATLRVDDHGTPIELLSALSHIHDSGVGKEGKKDHDTRKKLSGIARRARRLLHHRRTQLQQLDEVLRDLGFPIPTPGEFLDLNEQTDPYRVWRVRARLVEEKLPEELRGPAISMAVRHIARHRGWRNPYSKVESLLSPAEESPFMKALRERILATTGEVLDDGITPGQAMAQVALTHNISMRGPEGILGKLHQSDNANEIRKICARQGVSPDVCKQLLRAVFKADSPRGSAVSRVAPDPLPGQGSFRRAPKCDPEFQRFRIISIVANLRISETKGENRPLTADERRHVVTFLTEDSQADLTWVDVAEKLGVHRRDLRGTAVHTDDGERSAARPPIDATDRIMRQTKISSLKTWWEEADSEQRGAMIRYLYEDPTDSECAEIIAELPEEDQAKLDSLHLPAGRAAYSESLTALSDHMLATTDDLHEARKRLFGVDDSWAPPAEAINAPVGNPSVDRTLKIVGRYLSAVESMWGTPEVIHVEHVRDGFTSERMADERDKANRRRYNDNQEAMKKIQRDYGKEGYISRGDIVRLDALELQGCACLYCGTTIGYHTCQLDHIVPQAGPGSNNRRGNLVAVCERCNRSKSNTPFAVWAQKCGIPHVGVKEAIGRVRGWRKQTPNTSSEDLTRLKKEVIARLRRTQEDPEIDERSMESVAWMANELHHRIAAAYPETTVMVYRGSITAAARKAAGIDSRINLIGEKGRKDRIDRRHHAVDASVVALMEASVAKTLAERSSLRGEQRLTGKEQTWKQYTGSTVGAREHFEMWRGHMLHLTELFNERLAEDKVYVTQNIRLRLSDGNAHTVNPSKLVSHRLGDGLTVQQIDRACTPALWCALTREKDFDEKNGLPAREDRAIRVHGHEIKSSDYIQVFSKRKKTDSDRDETPFGAIAVRGGFVEIGPSIHHARIYRVEGKKPVYAMLRVFTHDLLSQRHGDLFSAVIPPQSISMRCAEPKLRKAITTGNATYLGWVVVGDELEINVDSFTKYAIGRFLEDFPNTTRWRICGYDTNSKLTLKPIVLAAEGLENPSSAVNEIVELKGWRVAINVLTKVHPTVVRRDALGRPRYSSRSNLPTSWTIE

SEQ ID NO: 19 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAAIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKTGETVRPLQSTRD

SEQ ID NO: 20 - protein, 6 aa, Artificial Sequence
  Description of Artificial Sequence Synthetic 6xHis tag
HHHHHH